(19) United States
(12) Patent Application Publication
(10) Pub. No.: US 2005/0191327 A1
(43) Pub. Date: Sep. 1, 2005

(54) Cosmetic composition comprising two different hetero polymers and method of using same

(76) Inventors: Wei Yu, Edison, NJ (US); Veronique Ferrari, Maisons-Alfort (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 11/019,382

(22) Filed: Dec. 23, 2004

Related U.S. Application Data
(60) Provisional application No. 60/531,968, filed on Dec. 24, 2003.

(51) Int. Cl. A61K 6/00; A61K 7/00

(52) U.S. Cl. 424/401

(57) ABSTRACT

The present disclosure is drawn to compositions comprising i) at least one liquid fatty phase, ii) at least one first polymer comprising a polymer skeleton which comprises at least one hydrocarbon-based repeating unit comprising at least one heteroatom, and iii) at least one second polymer, different from the first polymer, comprising a) a polymer skeleton which comprises at least one hydrocarbon-based repeating unit comprising at least one heteroatom, and b) at least one of: at least one terminal fatty chain chosen from alkyl chains and alkenyl chains, wherein the at least one terminal fatty chain is bonded to the polymer skeleton via at least one linking group; and at least one pendant fatty chain chosen from alkyl chains and alkenyl chains, wherein the at least one pendant fatty chain is bonded to the polymer skeleton via at least one linking group, wherein the first polymer and the second polymer are each present in a sufficient amount to render the composition stable, and wherein the liquid fatty phase is structured by at least one of the first polymer and the second polymer. The composition may be in the form of stable sticks and may give a shiny deposit when applied.

This application claims the benefit of U.S. Provisional Application No. 60/531,968, filed Dec. 24, 2003, which is herein incorporated by reference.

The present disclosure relates to compositions comprising at least one liquid fatty phase and at least two polymers each comprising at least one heteroatom. According to one embodiment, the composition may be in the form of a stable composition.

One aspect of the present disclosure relates to a cosmetic composition comprising two different polymers each comprising at least one heteroatom and a liquid fatty phase. The ratio between the two polymers can be chosen so that the composition is stable. Also disclosed herein are methods for care of and making-up the skin, including the scalp, and/or for the lips or for other keratinous materials, such as keratinous fibers.

As used herein, “liquid fatty phase” refers to a fatty phase which is liquid at room temperature (25° C.) and at atmospheric pressure (760 mm Hg, i.e. 101 kPa) and which comprises at least one fatty substance, such as an oil, which is liquid at room temperature and not soluble in water. If the liquid fatty phase comprises two or more fatty substances, they may be mutually compatible, such that they form a homogeneous phase macroscopically. A liquid takes the form of the container in which it is poured.

In one embodiment, the liquid fatty phase of the composition may be structured with at least one of the above-mentioned polymers comprising at least one heteroatom, such that the liquid fatty phase may be gelled or rigidified with the polymer.

As used herein, the term “gelled liquid fatty phase” refers to a liquid fatty phase whose viscosity is increased by adding the at least one polymer, and which flows under its own weight over time.

As used herein, the term “rigidified” refers to a liquid fatty phase whose viscosity is increased by adding the at least one polymer, and which does not flow under its own weight over time.

Structured liquid fatty phases in various products are known in the art. For example, U.S. Pat. No. 5,783,657 describes structuring a composition by using a polyamide, such as, for example, in a stick form. However, such a stick composition is usually not mechanically and/or thermally stable. Indeed, a part of the oil comprising such a composition tends to go outside or exude from the stick. Further, when the stick is applied on the skin or lips, the stick may be broken.

The application WO 02/47608 discloses compositions comprising a liquid fatty phase structured by one polyamide with terminal fatty chains, and a wax.

The present inventors have found that the use of two polymers, each comprising a heteroatom, may result in a stable composition leading to a shiny deposit on keratinous material. As used herein, the term “keratinous material” includes skin, such as the scalp, nails, lips, and keratinous fibers, such as hair, eyebrows, and eyelashes.

As used herein, the term “about”, appearing before a number given as a melting point, refers to the range or natural variation in the melting point. The range or variation may be due to impurities, the crystalline nature of the material, and/or the measurement method and conditions.

As used herein, the expression “at least one” refers to one or more and thus includes individual components as well as mixtures/combinations.

In one embodiment, disclosed herein is a composition comprising
i) at least one liquid fatty phase,
ii) at least one first polymer comprising a polymer skeleton which comprises at least one hydrocarbon-based repeating unit comprising at least one heteroatom, and
iii) at least one second polymer, different from the first polymer, comprising
   a) a polymer skeleton which comprises at least one hydrocarbon-based repeating unit comprising at least one heteroatom, and
   b) at least one of:
      at least one terminal fatty chain chosen from alkyl chains and alkenyl chains, wherein the at least one terminal fatty chain is bonded to the polymer skeleton via at least one linking group; and
      at least one pendant fatty chain chosen from alkyl chains and alkenyl chains, wherein the at least one pendant fatty chain is bonded to the polymer skeleton via at least one linking group,
wherein the first polymer and the second polymer are each present in an amount sufficient to render the composition stable, and
wherein the liquid fatty phase is structured by at least one of the first polymer and the second polymer.

A second aspect of the present disclosure relates to a composition comprising
i) at least one liquid fatty phase,
ii) at least one first polymer comprising
   a) a polymer skeleton which comprises at least one hydrocarbon-based repeating unit comprising at least one heteroatom, and
   b) at least one of:
      at least one terminal fatty chain chosen from alkyl chains and alkenyl chains, wherein the at least one terminal fatty chain is bonded to the polymer skeleton via at least one linking group; and
      at least one pendant fatty chain chosen from alkyl chains and alkenyl chains, wherein the at least one pendant fatty chain is bonded to the polymer skeleton via at least one linking group, and
iii) at least one second polymer, different from the first polymer, comprising
   a) a polymer skeleton which comprises at least one hydrocarbon-based repeating unit comprising at least one heteroatom, and
   b) at least one of:
      at least one terminal fatty chain chosen from alkyl chains and alkenyl chains, wherein the at least one terminal fatty chain is bonded to the polymer skeleton via at least one linking group; and
      at least one pendant fatty chain chosen from alkyl chains and alkenyl chains, wherein the at least one pendant fatty chain is bonded to the polymer skeleton via at least one linking group,
wherein the first polymer and the second polymer are each present in an amount sufficient to render the composition stable, and
wherein the liquid fatty phase is structured by at least one of the first polymer and the second polymer.

A third aspect of the present disclosure relates to a composition comprising
i) at least one liquid fatty phase:
ii) at least one first polymer comprising
   a) a polymer skeleton which comprises at least one hydrocarbon-based repeating unit comprising at least one heteroatom, and
   b) at least one of:
      at least one terminal fatty chain chosen from alkyl chains and alkenyl chains, wherein the at least one terminal fatty chain is bonded to the polymer skeleton via at least one ester linking group; and
      at least one pendant fatty chain chosen from alkyl chains and alkenyl chains, wherein the at least one pendant fatty chain is bonded to the polymer skeleton via at least one ester linking group, and
iii) at least one second polymer, different from the first polymer, comprising
   a) a polymer skeleton which comprises at least one hydrocarbon-based repeating unit comprising at least one heteroatom, and
   b) at least one of:
      at least one terminal fatty chain chosen from alkyl chains and alkenyl chains, wherein the at least one terminal fatty chain is bonded to the polymer skeleton via at least one amide linking group; and
      at least one pendant fatty chain chosen from alkyl chains and alkenyl chains, wherein the at least one pendant fatty chain is bonded to the polymer skeleton via at least one amide linking group,
wherein the second polymer does not comprise an ester linking group.

In this embodiment, the linking group of the second polymer may be a tertiary amide group.

Also disclosed herein is a make-up composition comprising
i) at least one liquid fatty phase,
ii) at least one first polymer comprising
   a) a polymer skeleton which comprises at least one hydrocarbon-based repeating unit comprising at least one heteroatom, and
   b) at least one terminal fatty chain that is bonded to the polymer skeleton via at least one ester linking group; and
iii) at least one second polymer comprising
   a) a polymer skeleton which comprises at least one hydrocarbon-based repeating unit comprising at least one heteroatom, and
   b) at least one terminal fatty chain that is bonded to the polymer skeleton via at least one linking group different from an ester group.

Another aspect of the present disclosure provides an anhydrous composition comprising the first polymer, the second polymer, and a coloring agent.

The first polymer can be a structuring polymer of the liquid fatty phase. The second polymer can be a structuring polymer of the liquid fatty phase. In one embodiment, the at least one first polymer and/or the at least one second polymer may be present in an amount effective to provide structure to the fatty phase. The liquid fatty phase can be structured by one of the two polymers comprising a heteroatom, or by the two polymers at the same time.

The at least one first polymer and the at least one second polymer can be present in a combined amount to provide the composition with stability. In a further embodiment, the at least one first polymer and/or the at least one second polymer can provide resistance to shear.

In one embodiment, the at least one first polymer and the at least one second polymer provide the composition with stability and resistance to shear.

As defined herein, “stability” can be tested by placing a sample of the composition in a controlled environment chamber at 25° C. In this test, the physical condition of the sample can be inspected as it is placed in the chamber. The sample can then be inspected at 24 hours, 3 days, 1 week, 2 weeks, 4 weeks and 8 weeks. At each inspection, the sample can be examined for abnormalities in the composition such as:
i) bending when the composition is in a stick form (the stick is placed in a vertical position and if it bends under its own weight, it is not stable; bending can be visible to the naked eye, or a small ruler can be placed along the stick to provide a reference point to ascertain the leaning);
ii) melting of a solid composition, totally or partially; and/or
iii) phase separation (when the composition is in the liquid form, at least two phases appear in the container), or syneresis (when the composition is in the solid form). As used herein, “syneresis,” also called exudation, refers to the appearance of droplets that are visible to the naked eye on the surface of a solid composition.

The stability of the composition can be further tested by repeating any of the preceding tests in a controlled environment chamber at 4° C., 37° C., 45° C., 50° C. or under freeze-thaw conditions.

According to one embodiment, a composition may be considered to be stable if syneresis or exudation or phase separation does not appear before the end of a 8 week time period, in a controlled chamber at 25° C. In this embodiment, the composition may show no syneresis or exudation before the end of an 8 week time period at 45° C., or even at 50° C.

In a further embodiment, a composition in the form of a stick may be considered to be stable if no bending, as described above, is observed before the end of a 8 week time period at 25° C., or even at 45° C.

Furthermore, the skilled artisan will readily recognize an abnormality that impedes the functioning of a composition based on the intended application.

Another embodiment relates to a method for providing stability to a composition comprising a liquid fatty phase, by introducing
i) at least one first polymer comprising a polymer skeleton which comprises at least one hydrocarbon-based repeating unit comprising at least one heteroatom, and
ii) at least one second polymer comprising
   a) a polymer skeleton which comprises at least one hydrocarbon-based repeating unit comprising at least one heteroatom, and
   b) at least one of:
      at least one terminal fatty chain chosen from alkyl chains and alkenyl chains, bonded to the polymer skeleton via at least one linking group; and
      at least one pendant fatty chain chosen from alkyl chains and alkenyl chains, bonded to the polymer skeleton via at least one linking group,
wherein each fatty chain is present in a sufficient amount to render the composition stable.

For example, the polymer skeleton of the at least one first polymer and/or second polymer can comprise at least one terminal or pendant fatty chain wherein the at least one chain may be chosen from alkyl chains comprising at least four carbon atoms and alkenyl chains comprising at least four carbon atoms. The polymer skeleton may comprise at least one polyamide block or a polyamide polymer. In this embodiment, the fatty chains may be bonded to any carbon or nitrogen of the polyamide skeleton, via at least one ester linking group. The at least one linking group can also be chosen from ether, polyether, tertiary amide and secondary amide groups.

It is to be understood that both the foregoing general description and the following detailed description are exemplary and explanatory only and are not restrictive of the invention as claimed. Other embodiments will be apparent to those skilled in the art from consideration of the specification and practice of the invention disclosed herein.

One aspect of the present disclosure relates to cosmetic compositions which are useful for the care, make-up and/or treating of at least one keratinous material, including at least one keratinous fiber, and nails. These compositions may be of suitable hardness to allow preparation of these compositions in the form of a stick or other structured stable forms, such as pastes, gels or dishes.

The disclosed compositions apply not only to make-up products for at least one keratinous material such as lip compositions, lip pencils, foundations, including foundations which may be cast in the form of a stick or a dish, concealer products, temporary tattoo products, eyeliners, mascara bars, but also to body hygiene products such as deodorant sticks, and to care products and products for treating at least one keratinous material, such as sunscreen and anti-sun products which may be in stick form. Further embodiments include compositions in the form of mascara products including mascara bars, eyeliner products, foundation products, lipstick products, blush products for cheeks or eyelids, deodorant products, make-up products for the body, make-up-removing products, eyeshadow products, face powder products, concealer products, treating shampoo products, nail varnish products, hair conditioning products, sunscreen products, colorant products for the skin or hair, or skin care formulas such as, for example, anti-pimple or shaving cut formulas. As defined herein, a deodorant product refers to a personal hygiene product and does not relate to care, make-up or treatment of keratin materials, including keratin fibers and nails.

For example, compositions of the present disclosure may be in a form chosen from a paste, a solid, a gel, and a cream. The compositions may be in a form chosen from an emulsion, i.e., an oil-in-water or water-in-oil emulsion; a multiple emulsion, e.g., an oil-in-water-in-oil emulsion or water-in-oil-in-water emulsion; or a solid, rigid or supple gel, including anhydrous gels. In one embodiment, the compositions may be anhydrous. The compositions may, for example, comprise an external or continuous fatty phase.

In another embodiment, the compositions may be transparent or clear. The compositions can also be in a form chosen from a translucent anhydrous gel and a transparent anhydrous gel.

The compositions can also be molded or cast as a stick or a dish. The compositions in one embodiment may comprise a solid form such as a molded stick or a poured stick.

The structuring of the liquid fatty phase can be modified according to the nature of the first polymer, the nature of the second polymer, the amount of the first polymer and the amount of the second polymer, and may be such that a rigid structure, in the form of a rod or stick with good mechanical strength, is obtained. When these rods or sticks are colored, they may make it possible, after application, to obtain a uniformly colored glossy deposit which does not migrate and which has good staying power or long-wearing properties, for example, of the color, over time.

In one embodiment, the presently disclosed composition is a composition for the lips, such as a lipstick composition or lip gloss.

The presently disclosed compositions can be essentially free of hydrocarbon wax, wherein “essentially free” means that the presence of the hydrocarbon wax does not materially affect the properties of the composition. In one embodiment, the compositions may be free of wax. As used herein, the term “free of wax” means less than 10%, such as less than 5%, and further such as less than 3% by weight of wax, or, for example, having no wax at all.

As used herein, “wax” refers to a solid at ambient temperature (25° C.) having a sharp, well-defined reversible solid to liquid transition between 30° C. and 200° C., and having in the solid state an anisotropic crystalline organization. The crystal facets of the wax are such that the crystals diffract and/or diffuse light, making a composition comprising a wax look cloudy, e.g., more or less opaque. When the wax is brought to its melting temperature, it may be possible to mix it with a continuous fatty phase and to effect a homogeneous mixture. However, when the temperature is returned to ambient temperature, re-crystallization of the wax in the oils of the mixture occurs. This re-crystallisation is believed to be responsible for both the mixture's structure and also for its reduction in gloss.

One aspect of the present disclosure provides compositions that are essentially free of wax, meaning that the compositions do not comprise a sufficient quantity of wax to noticeably impact the structuring of the composition. In another aspect, the compositions comprise no wax.

The waxes that may be used herein may include those of natural origin such as beeswax, Carnauba wax, Candelilia wax, Ouricoury wax, Japan wax, cork or sugar cane fibres, paraffin, lignite waxes, lanolin wax, Montan wax, ozokerites, hydrogenated oils such as hydrogenated jojoba oil, synthetically produced waxes such as polyethylene wax, resulting from the polymerisation of ethylene, waxes obtained by Fischer-Tropsch synthesis, microcrystalline waxes, the esters of fatty acids and glycerides, and the silicone waxes such as alkyl, alkoxy and/or esters of poly(di)methyl siloxane, which are solid at 40° C.

First polymer

The at least one first polymer may be a solid that is not deformable at room temperature (25° C.) and atmospheric pressure (760 mm Hg, i.e., 101 kPa). In one embodiment, the at least one first polymer may be capable of structuring the composition. In another embodiment, the at least one first polymer may structure the composition without opacifying it.

As defined above, the at least one first polymer comprises a polymer skeleton comprising at least one hydrocarbon-based repeating unit comprising at least one heteroatom.

The at least one first polymer, for example, may comprise a polymer skeleton that comprises at least one hydrocarbon-based repeating unit comprising at least one heteroatom.

In one embodiment, the at least one first polymer may comprise a) a polymer skeleton which comprises at least one hydrocarbon-based repeating unit comprising at least one heteroatom and b) at least one terminal or pendant fatty chain. The at least one first polymer may have a softening point ranging from 70° C. to 100° C. The softening point can be measured by a well known method such as “Differential Scanning Calorimetry” (i.e., DSC method) with a temperature rise ranging from 5 to 10° C./min or the Ring and Ball method. The at least one first polymer may be a non-waxy polymer.

The at least one first polymer may be present in the composition in an amount ranging from 0.5% to 80% by weight relative to the total weight of the composition, such as ranging from 2% to 60%, from 5% to 40%, from 5% to 25%, and further from 5% to 15%.

First polymer Comprising a heteroatom

In one embodiment, the composition comprises at least one first polymer comprising a polymer skeleton, which comprises at least one hydrocarbon-based repeating unit comprising at least one heteroatom.

Non-limiting examples of the at least one first polymer that may be used in this embodiment include polyamide polymers (or polyamide resins) resulting from the condensation of at least one aliphatic dicarboxylic acid and at least one diamine, the carbonyl and amine groups being condensed via an amide bond. Examples of these polyamide polymers include, but are not limited to, those sold or made under the brand name VERSAMID by the companies General Mills, Inc., and Henkel Corp. (VERSAMID 930, 744 or 1655) or by the company Olin Mathieson Chemical Corp. under the brand name ONAMID (ONAMID S or C). These resins have a weight-average molecular mass ranging from 6000 to 9000. For further information regarding these polyamides, reference may be made to U.S. Pat. Nos. 3,645,705 and 3,148,125, the disclosures of which are herein incorporated by reference.

Other examples of polyamides include those sold by the company Arizona Chemical under the references UNI-REZ (2658, 2931, 2970, 2621, 2613, 2624, 2665, 1554, 2623 and 2662) and the product sold or made under the reference MACROMELT 6212 by the company Henkel. For further information regarding these polyamides, reference may be made to U.S. Pat. No. 5,500,209, the disclosure of which is herein incorporated by reference. Such polyamides may display high melt viscosity characteristics. MACROMELT 6212, for example, has a high melt viscosity at 190° C. of 30-40 poise (as measured by a Brookfield Viscometer, Model RVF #3 spindle, 20 RPM).

The at least one first polymer may be chosen from polyamide resins from vegetable sources. Polyamide resins from vegetable sources may be chosen from, for example, the polyamide resins of U.S. Pat. Nos. 5,783,657 and 5,998,570, the disclosures of which are herein incorporated by reference.

In one embodiment, when the at least one first polymer comprises a urea urethane having the following formula (I):
R—O—CO—NH—R′—NH—CO—NH—R″—NH—CO—NH—R′—NH—CO—OR (I)
then R is chosen from
CₙH₂ₙ₊₁—, wherein n is an integer having a value greater than 22, for example from 23 to 120, and further, for example, from 23 to 68; and
CₘH₂ₘ₊₁(OCₚH₂ₚ)ᵣ—, wherein m is an integer having a value of greater than 18, for example, from 19 to 120, and further, for example, from 23 to 68, p is an integer having a value of from 2 to 4, and r is an integer having a value of from 1 to 10.
R′ is chosen from: 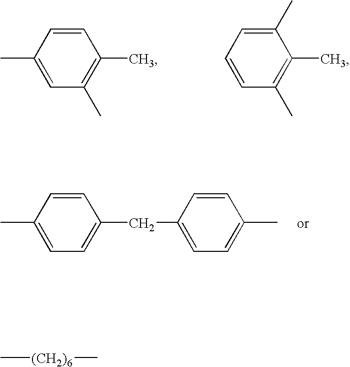
   and R″ is chosen from:

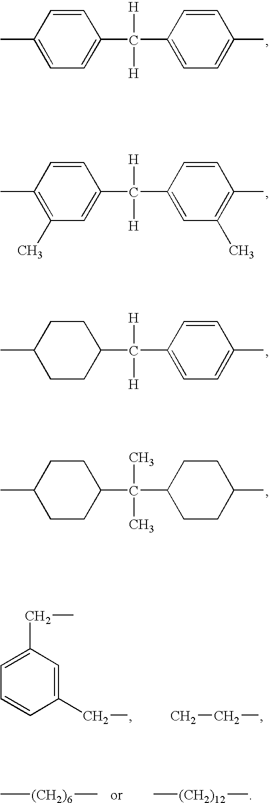

In another embodiment, the at least one first polymer is not a urea urethane of the formula (I):
R—O—CO—NH—R′—NH—CO—NH—R″—NH—CO—NH—R′—NH—CO—OR (I)
wherein R is chosen from CₙH₂ₙ₊₁— and CₘH₂ₘ₊₁(CₚH₂ₚO)ᵣ—; and wherein n is an integer having a value of from 4 to 22; m is an integer having a value of from 1 to 18; p is an integer having a value of from 2 to 4; and r is an integer having a value of from 1 to 10.
R′ is chosen from: 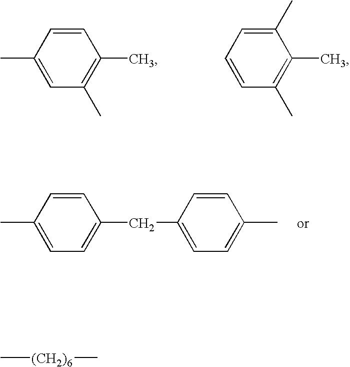
   and R″ is chosen from:

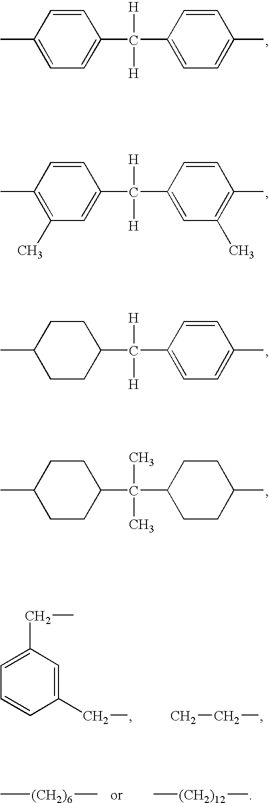

The at least one first polymer may have a softening point greater than 50° C., such as from 65° C. to 190° C., for example, from 70° C. to 150° C., such as from 70° C. to 130° C., and further, such as from 80° C. to 100° C. This softening point may be lower than that of structuring polymers used in the art, which may facilitate the use of the at least one first polymer of the present disclosure and may limit the degradation of the liquid fatty phase. These polymers may be non-waxy polymers. The softening point can be measured by a well known method such as “Differential Scanning Calorimetry” (i.e., DSC method) with a temperature rise of 5 to 10° C./min, or Ring and Ball method.

First and/or Second polymer with at Least One Terminal and/or Pendant Fatty Chain

In one embodiment, the at least one first polymer and/or second polymer can comprise at least one terminal fatty chain chosen from alkyl and alkenyl chains, comprising, for instance, at least 4 atoms, and, further for instance, comprising 8 to 120 carbon atoms, bonded to the polymer skeleton via at least one linking group. The terminal fatty chain may, for example, be functionalized. The at least one first polymer and/or second polymer may also further comprise at least one pendant fatty chain chosen from alkyl and alkenyl chains, comprising, for instance, at least 4 atoms, and, for example, comprising 8 to 120 carbon atoms, bonded to any carbon or heteroatom of the polymer skeleton via at least one linking group. The pendant fatty chain may, for example, be functionalized. The at least one first and/or second polymer may comprise both at least one pendant fatty chain and at least one terminal fatty chain as defined above, and each type of chain can independently be functionalized.

In one embodiment, the first polymer and/or second polymer comprises at least two hydrocarbon-based repeating units. In another embodiment, the first polymer and/or second polymer comprises at least three hydrocarbon-based repeating units and as a further example, the at least three repeating units may be identical.

As used herein, “functionalized” means comprising at least one functional group. Non-limiting examples of functional groups include hydroxyl groups, ether groups, oxyalkylene groups, polyoxyalkylene groups, carboxylic acid groups, amine groups, amide groups, halogen containing groups, including fluoro and perfluoro groups, halogen atoms, ester groups, siloxane groups and polysiloxane groups.

As used herein, the expression “functionalized chain” means, for example, an alkyl chain comprising at least one functional (reactive) group chosen, for example, from those recited above. For example, in one embodiment, the hydrogen atoms of at least one alkyl chain may be substituted at least partially with fluorine atoms.

According to one embodiment, these chains may be linked directly to the polymer skeleton, or via a linking group chosen from an ester group and a perfluoro group.

As used herein, the term “polymer” means a compound comprising at least two repeating units, such as, for example, a compound comprising at least three repeating units, which may be identical.

As used herein, the expression “hydrocarbon-based repeating unit” includes a repeating unit comprising from 2 to 80 carbon atoms, such as, for example, from 2 to 60 carbon atoms. The at least one hydrocarbon-based repeating unit may also comprise oxygen atoms. The hydrocarbon-based repeating unit may be chosen from saturated and unsaturated hydrocarbon-based repeating units, which in turn may be chosen from linear hydrocarbon-based repeating units, branched hydrocarbon-based repeating units and cyclic hydrocarbon-based repeating units. The at least one hydrocarbon-based repeating unit may comprise, for example, at least one heteroatom that is part of the polymer skeleton, i.e., not pendant. The at least one heteroatom may be chosen, for example, from nitrogen, sulfur, and phosphorus. For example, the at least one heteroatom may be a nitrogen atom, such as a non-pendant nitrogen atom. In another embodiment, the at least one hydrocarbon-based repeating unit may comprise at least one heteroatom, with the proviso that the at least one heteroatom is not nitrogen. In another embodiment, the at least one heteroatom may be combined with at least one atom chosen from oxygen and carbon to form a heteroatom group. In one embodiment, the heteroatom group comprises a carbonyl group.

The at least one hydrocarbon-based repeating unit comprising at least one heteroatom group may be chosen, for example, from amide groups, carbamate groups, and urea groups. In one embodiment, the at least one repeating unit comprises amide groups forming a polyamide skeleton. In another embodiment, the at least one repeating unit comprises carbamate groups and/or urea groups forming a polyurethane skeleton, a polyurea skeleton and/or a polyurethane-polyurea skeleton. The pendant chains, for example, can be linked directly to at least one of the heteroatoms of the polymer skeleton. In yet another embodiment, the at least one hydrocarbon-based repeating unit may comprise at least one heteroatom group with the proviso that the at least one heteroatom group is not an amide group. In one embodiment, the polymer skeleton comprises at least one repeating unit chosen from silicone units and oxyalkylene units, the at least one repeating unit being between the hydrocarbon-based repeating units.

In one embodiment, the composition disclosed herein comprises a) at least one first polymer and/or second polymer comprising at least one hydrocarbon-based repeating unit comprising at least one nitrogen atom, such as amide, urea, or carbamate units, further such as amide units, and b) at least one polar oil.

In one embodiment, in the at least one first polymer and/or second polymer, the percentage of the total number of fatty chains ranges from 40% to 98% relative to the total number of repeating units and fatty chains, for example, from 50% to 95%. In a further embodiment wherein the polymer skeleton is a polyamide skeleton, in the at least one first polymer and/or second polymer, the percentage of the total number of fatty chains ranges from 40% to 98% relative to the total number of all amide units and fatty chains, for example, from 50% to 95%.

First and/or Second polyamide polymer with at Least One Terminal and/or Pendant Fatty Chain

In another embodiment, the at least one first polymer and/or second polymer comprises a polyamide comprising a polymer skeleton, wherein at least one amide repeating unit, and optionally at least one pendant fatty chain and/or at least one terminal fatty chain, may be optionally functionalized and comprise from 8 to 120 carbon atoms, bonded to at least one of the amide repeating units via at least one ester linking group. When the first or second polymer has amide repeating units, the pendant fatty chains may be linked to at least one of the nitrogen atoms in the amide repeating units.

In one embodiment, the at least one first polymer and/or second polymer, for example, the polyamide polymer, may have a weight-average molecular mass of less than 100,000, such as less than 50,000. In another embodiment, the weight-average molecular mass may range from 1000 to 30,000, such as from 2000 to 20,000, and further such as from 2000 to 10,000.

In another embodiment, the weight-average molecular mass may range up to 500,000, and further up to 1,000,000.

The at least one first polymer and/or second polymer, for example, the polyamide polymer, may be non-soluble in water or in an aqueous phase. In another embodiment, the at least one first and/or second polymer may have a non-ionic group.

In one embodiment, the at least one first polymer and/or second polymer may, for example, be chosen from polyamide polymers comprising at least one polyamide skeleton with
a) at least one terminal fatty chain chosen from alkyl chains, for example, alkyl chains comprising at least four carbon atoms, and alkenyl chains, for example, alkenyl chains comprising at least four carbon atoms, bonded to the at least one polyamide skeleton via at least one linking group, and/or
b) at least one pendant fatty chain chosen from alkyl chains, for example, alkyl chains comprising at least four carbon atoms, and alkenyl chains, for example, alkenyl chains comprising at least four carbon atoms, bonded to the at least one polyamide skeleton via at least one linking group.

In one embodiment, the at least one polyamide skeleton may comprise at least one terminal fatty chain chosen from fatty chains comprising 8 to 120 carbon atoms, such as, for example, 12 to 68 carbon atoms, bonded to the at least one polyamide skeleton via at least one linking group and/or at least one pendant fatty chain chosen from fatty chains comprising 8 to 120 carbon atoms, such as, for example, 12 to 68 carbon atoms, bonded to the at least one polyamide skeleton via at least one linking group, such as bonded to any carbon or nitrogen of the polyamide skeleton via the at least one linking group. In one embodiment, the at least one linking group may be chosen from single bonds and urea, urethane, thiourea, thiourethane, thioether, thioester, ester, ether and amine groups. The linking group may be, for example, an ester group. In one embodiment, these polymers may comprise a fatty chain at each end of the polymer skeleton, such as the polyamide skeleton.

As used herein, a composition may be referred to as soluble if the composition has a solubility of greater than 0.01 g per 100 mL of solution at 25° C. In one embodiment, due to the presence of at least one fatty chain, the polyamide polymers may be readily soluble in oils (i.e., water-immiscible liquid compounds) and thus may give macroscopically homogeneous compositions. In a further embodiment, a high content (at least 25%) of the polyamide polymers may be readily soluble in oils and thus may give macroscopically homogeneous compositions, unlike certain polymers of the prior art that do not comprise such alkyl or alkenyl chains at the end of the polyamide skeleton.

In a further embodiment, the polyamide polymers can be chosen from polymers resulting from at least one polycondensation reaction between at least one acid chosen from dicarboxylic acids comprising at least 32 carbon atoms, such as 32 to 44 carbon atoms, and at least one amine chosen from diamines comprising at least 2 carbon atoms, such as from 2 to 36 carbon atoms, and triamines comprising at least 2 carbon atoms, such as from 2 to 36 carbon atoms. The at least one dicarboxylic acid can, for example, be chosen from dimers of at least one fatty acid comprising at least 16 carbon atoms, such as oleic acid, linoleic acid and linolenic acid. The at least one amine can, for example, be chosen from diamines, such as ethylenediamine, hexylenediamine, hexamethylenediamine, phenylenediamine, and triamines, such as ethylenetriamine.

The polyamide polymers may also be chosen from polymers comprising at least one terminal carboxylic acid group. The at least one terminal carboxylic acid group can, for example, be esterified with at least one alcohol chosen from monoalcohols comprising at least 4 carbon atoms. For example, the at least one alcohol can be chosen from monoalcohols comprising from 10 to 36 carbon atoms. In a further embodiment, the monoalcohols can comprise from 12 to 24 carbon atoms, such as from 16 to 24 carbon atoms, and for example, 18 carbon atoms.

The second polymer may be chosen from polyamide polymers and polyamide block copolymers of formula (II) 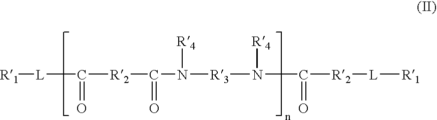
wherein:
n is an integer from 1 to 30,
R′₁, which may be identical or different, each represent a fatty chain and are each independently chosen from alkyl groups comprising at least one carbon atom and alkenyl groups comprising at least four carbon atoms;
R′₂, which may be identical or different, are each independently chosen from C₁ to C₅₂ hydrocarbon diradicals;
R′₃, which may be identical or different, are each independently chosen from organic groups comprising atoms chosen from carbon atoms, hydrogen atoms, oxygen atoms and nitrogen atoms, with the proviso that R′₃ comprises at least 2 carbon atoms;
R′₄, which may be identical or different, are each independently chosen from hydrogen atoms, C₁ to C₁₀ alkyl groups and a direct bond to at least one group chosen from R′₃ and another R′₄, such that when the at least one group is chosen from another R′₄, the nitrogen atom to which both R′₃ and R′₄ are bonded forms part of a heterocyclic structure defined in part by R′₄—N—R′₃, with the proviso that at least 50% of all R′₄ are chosen from hydrogen atoms; and
L represents the linking group described above, which may be substituted by at least one R′₁ group as described above. In one embodiment, L may be a group of formula:

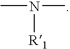

Ester Terminated polyamide

In one embodiment, the at least one polyamide polymer may be chosen from those described in U.S. Pat. No. 5,783,657, the disclosure of which is incorporated herein by reference, which include polymers of formula (III): 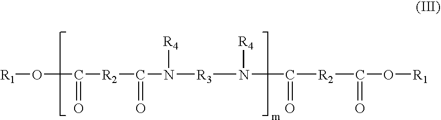
wherein:
m is an integer which represents the number of amide units such that the number of ester groups present in the at least one polyamide polymer ranges from 10% to 50% of the total number of all the ester groups and all the amide groups comprised in the at least one polyamide polymer;
R₁, which may be identical or different, are each independently chosen from alkyl groups comprising at least 4 carbon atoms and alkenyl groups comprising at least 4 carbon atoms. In one embodiment, the alkyl group comprises from 4 to 24 carbon atoms and the alkenyl group comprises from 4 to 24 carbon atoms;
R₂, which may be identical or different, are each independently chosen from C₄ to C₄₂ hydrocarbon-based groups, with the proviso that at least 50% of all R₂ groups are chosen from C₃₀ to C₄₂ hydrocarbon-based groups;
R₃, which may be identical or different, are each independently chosen from organic groups comprising at least two carbon atoms, in addition to hydrogen atoms, and optionally comprising at least one atom chosen from oxygen atoms and nitrogen atoms; and
R₄, which may be identical or different, are each independently chosen from hydrogen atoms, C₁ to C₁₀ alkyl groups and a direct bond to at least one group chosen from R₃ and another R₄ such that when the at least one group is chosen from another R₄, the nitrogen atom to which both R₃ and R₄ are bonded forms part of a heterocyclic structure defined in part by R₄—N—R₃, with the proviso that at least 50% of all R₄ are chosen from hydrogen atoms.

In one embodiment, at least one of the terminal fatty chains of formula (III) may be linked to the last heteroatom, in this case nitrogen, of the polyamide skeleton. In a further embodiment, the terminal chains may be functionalized. In another embodiment, the ester groups of formula (III), linked to the terminal and/or pendant fatty chains, may represent from 15% to 40% of the total number of ester and amide groups (i.e., heteroatom groups), such as, for example, from 20% to 35%.

In one embodiment, m may be an integer ranging from 1 to 10, for example from 1 to 5, and as a further example, an integer ranging from 3 to 5. In another embodiment, R₁, which may be identical or different, can each independently be chosen from C₁₂ to C₂₂ alkyl groups, such as from C₁₆ to C₂₂ alkyl groups.

For example, R₂, which may be identical or different, can each independently be chosen from C₁₀ to C₄₂ alkyl groups. In one embodiment, at least 50% of all R₂, which may be identical or different, can, for example, each be independently be chosen from groups comprising from 30 to 42 carbon atoms. In another embodiment, at least 75% of all R₂, which may be identical or different, can, for example, each be independently be chosen from groups comprising from 30 to 42 carbon atoms. In the two aforementioned embodiments, the remaining R₂, which may be identical or different, can, for example, each independently be chosen from C₄ to C₁₉ groups, such as C₄ to C₁₂ groups.

R₃, which can be identical or different, can, for example, each independently be chosen from C₂ to C₃₆ hydrocarbon-based groups and polyoxyalkylene groups. In another embodiment, R₃, which can be identical or different, can each, for example, be chosen from C₂ to C₁₂ hydrocarbon-based groups. In another embodiment, R₄, which can be identical or different, can each independently be chosen from hydrogen atoms.

As used herein, “hydrocarbon-based groups” may be chosen from linear, cyclic and branched, and saturated and unsaturated groups. The hydrocarbon-based groups can be chosen from aliphatic and aromatic groups. In one embodiment, the hydrocarbon-based groups may be chosen from aliphatic groups. As used herein, the alkyl and alkylene groups may be chosen from linear, cyclic and branched, and saturated and unsaturated groups.

As used herein, the pendant and terminal fatty chains may be chosen from linear, cyclic and branched, and saturated and unsaturated groups. The pendant and terminal fatty chains can be chosen from aliphatic and aromatic groups. In one embodiment, the pendant and terminal fatty chains may be chosen from aliphatic groups.

An aspect of the present disclosure includes structuring the liquid fatty phase with the aid of at least one first polymer, such as the at least one polymer of formula (III). The at least one polyamide polymer of formula (III) may, for example, be in the form of a mixture of polymers, and this mixture may also comprise a compound of formula (III) wherein m is equal to zero, i.e. a diester.

Non-limiting examples of at least one ester-terminated polyamide polymer include the commercial products sold or made by Arizona Chemical under the names Uniclear 80 and Uniclear 100, which can be ethylenediamine stearyl dimer tallate or dilinoleate copolymers. These polymer products are sold, respectively, in the form of an 80% (in terms of active material) gel in a mineral oil and a 100% (in terms of active material) gel. These polymers may have a softening point ranging from 88° C. to 94° C., may be mixtures of copolymers derived from monomers of (i) C₃₆ diacids and (ii) ethylenediamine, and may have a weight-average molecular mass of about 6000. Terminal ester groups may result from esterification of the remaining acid end groups with at least one alcohol chosen from cetyl alcohol and stearyl alcohol. A mixture of cetyl and stearyl alcohols may be called cetylstearyl alcohol.

Ester Terminated poly(ester-amide)

In one embodiment, the at least one first polymer and/or second polymer can be an ester-terminated poly(ester-amide) (ETPEA).

In another embodiment, the second polymer may be an ester-terminated poly(ester-amide) polymer and the first polymer may be an ester terminated polyamide as described above.

An exemplary ETPEA polymer can be a resin composition prepared by reacting components comprising dibasic acid, diamine, polyol and mono-alcohol, wherein at least 50 equivalent percent of the dibasic acid comprises polymerized fatty acid, and at least 50 equivalent percent of the diamine comprises ethylenediamine. In other words, polymerized fatty acid contributes at least 500/% of the diacid equivalents present in the reaction mixture, and ethylenediamine contributes at least 50% of the diamine equivalents present in the reaction mixture.

The resin composition can be prepared by reacting components comprising dibasic acid, diamine, polyol and monoalcohol, wherein
i) at least 50 equivalent percent of the dibasic acid comprises polymerized fatty acid; and
ii) at least 50 equivalent percent of the diamine comprises ethylenediamine.

In one embodiment, 10 to 60 equivalent percent of the total of the hydroxyl and amine equivalents provided by diamine, polyol and monoalcohol may be provided by monoalcohol; and no more than 50 equivalent percent of the total of the hydroxyl and amine equivalents provided by diamine, polyol and monoalcohol may be provided by polyol.

A method for preparing a resin composition comprising ester-terminated poly(ester-amide) is described in U.S. Pat. No. 6,552,160, which is herein incorporated by reference.

As used herein, dibasic acid refers to an organic molecule comprising two carboxylic acid groups or reactive equivalents thereof. In one embodiment, the dibasic acid -may be a polymerized fatty acid, such as the dimer acid component of polymerized fatty acid. As used herein, polymerized fatty acid refers to a mixture of structures, including dimer acid and trimer acid, wherein individual dimer acids may be saturated, unsaturated, cyclic, acyclic, etc. Polymerized fatty acid as used to form the resin of the ETPEA is a well known material of commerce, and thus need not be described in great detail. Polymerized fatty acid may be formed by heating long-chain unsaturated fatty acids, e.g., C₁₈ monocarboxylic acids, to about 200-250° C. in the presence of a clay catalyst so that the fatty acids polymerize. The polymerized fatty acid may comprise dimer acid, for example, C₃₆ dicarboxylic acid formed by dimerization of the fatty acid, and trimer acid, for example, C₅₄ tricarboxylic acid formed by trimerization of the fatty acid. A more detailed discussion of fatty acid polymerization may be found in, e.g., U.S. Pat. No. 3,157,681 and Naval Stores—Production, Chemistry and Utilization, D. F. Zinkel and J. Russell (Eds.), Pulp. Chem. Assoc., Inc., 1989, Chapter 23.

In addition to polymerized fatty acid, or reactive equivalents thereof, the dibasic acid may comprise dibasic acid of the formula HOOC—R₁—COOH. The variable R₁ may be aliphatic or aromatic.

The diamine reactant has two amine groups, both of which may be primary amines, and may be represented by the formula HN(R₂ₐ)—R₂−N(R₂ₐ)H. In one embodiment, R₂ₐ may be hydrogen. In another embodiment, R₂ₐ may be an alkyl group. In a further embodiment, R₂ₐ may be joined together with R₂ or another R₂ₐ to form a heterocyclic structure. In one embodiment, the diamine may be ethylenediamine, i.e., a diamine wherein R₂ₐ is hydrogen and R₂ is —CH₂—CH₂—.

Diamines other than ethylenediamine may be referred to herein as co-diamines. When present, co-diamines may be used in a minor amount compared to the ethylenediamine.

The monoalcohol may be represented by the formula R₃—OH, wherein R₃ may be a hydrocarbon group comprising at least ten carbon atoms. Thus, the monoalcohol can also be described as a monohydric alcohol. In one aspect of the present disclosure, R₃ may be a C₁₀₋₃₀ hydrocarbon, such as a C₁₂₋₂₄ hydrocarbon, and further such as a C₁₆₋₂₂ hydrocarbon. In one embodiment, R₃ may be a C₁₈ hydrocarbon. As used herein, the term C₁₀₋₃₀ hydrocarbon refers to a hydrocarbon group comprising at least 10, but not more than 30 carbon atoms, and similar terms have an analogous meaning. The carbon atoms of the hydrocarbon group may be arranged in a linear, branched or cyclic fashion, and the group may be saturated or unsaturated.

In one aspect of the present disclosure, R₃ may be linear, with the hydroxyl group located on a terminal carbon atom, i.e., the monoalcohol may be a primary monoalcohol. Non-limiting examples of monoalcohols for preparing ETPEA resins include 1-dodecanol, decanol, 1-tetradecanol, 1-hexadecanol (cetyl alcohol), 1-octadecanol (stearyl alcohol), 1-eicosanol (arachidyl alcohol) and 1-docosanol (behenyl alcohol), where the names in parentheses are common or trivial names by which these monoalcohols are known. In another embodiment, the monoalcohol may comprise an alkenyl group, i.e., an alkyl group having unsaturation between at least any two adjacent carbon atoms.

Another monoalcohol reactant is a so-called Guerbet alcohol. Guerbet alcohols have the general formula H—C(Rₐ)(R_{b})CH₂—OH, wherein Rₐ and R_{b} may be the same or different and, in one embodiment, may represent a C₆₋₁₂ hydrocarbon group. Further discussion of Guerbet alcohols may be found in, e.g., “Dictionary For Auxiliaries For Pharmacy, Cosmetics And Related Fields,” H. P. Fiedler, 3rd Ed., 1989, Cantor Aulendorf. In one embodiment, a Guerbet alcohol may be 2-hexadecyloctadecanol, which comprises 24 carbon atoms.

In another embodiment, the monoalcohol may be a linear wax alcohol. Suitable linear wax alcohols may be commercially available from, e.g., Petrolite Corporation (Tulsa, Okla.) under their UNILIN® trademark. The linear wax alcohols may be a blend of linear alcohols comprising at least 20 carbon atoms, such as at least 24 carbon atoms. In one embodiment, the linear wax alcohol may comprise from 22 to 70 carbon atoms. Vapor pressure osmometry (VPO), among many other techniques, may be used to characterize the average molecular weight of a blend of alcohols. In one aspect, the mixture of monohydric linear wax alcohols may have an average molecular weight by VPO from 200 to 800, further from 300 to 600. Pure C₂₂ monohydric linear alcohol has a molecular weight of 326 by VPO.

The monohydric alcohol, whether present as a substantially pure alcohol or in a mixture of monohydric alcohols, may have a straight chain alkyl group. Non-limiting exemplary alcohols include 1-eicosanol (C₂₀), 1-docosanol (C₂₂, also known as behenyl alcohol), dotriacontanol (C₃₂), tetratriacontanol (C₃₄), pentatriacontanol (C₃₅), tetracontanol (C₄₀), tetraacontanol (C₄₄), dopentaacontanol (C₅₄), tetrahexaacontanol (C₆₄), and dohexaacontanol (C₇₂).

Another component used in preparing an ETPEA resin is polyol, which may also be referred to as polyhydric alcohol. The polyol may be of the formula R₄—(OH)ₙ wherein R₄ is an n-valent organic group. For instance, R₄ may be a C₂-C₂₀ organic group without hydroxyl substitution. As another example, R₄ may be a hydrocarbon. In one embodiment, n may be chosen from 2, 3, 4, 5 and 6. Non-limiting exemplary polyols for use in preparing an ETPEA resin include ethylene glycol, propylene glycol, butylene glycol, glycerol, trimethylolpropane, pentaerythritol, neopentyl glycol, tris(hydroxylmethyl)methanol, di-pentaerythritol, and tri-pentaerthyritol.

Reactive equivalents of diacids and/or diamines may be used to prepare ETPEA resin. For example, diesters may be substituted for some or all of the diacid. As used herein, the term “diesters” refers to the esterification product of a diacid with molecules comprising at least one hydroxyl group. Such diesters may be prepared from relatively volatile molecules comprising at least one hydroxyl group, in order that the molecule comprising at least one hydroxyl group may be easily removed from the reaction vessel subsequent to monoalcohol and/or diamine (both as defined herein) reacting with the diester. A lower alkyl diester, e.g., the esterification or diesterification product of a diacid as defined herein and a C₁₋₄ monohydric alcohol (e.g., methanol, ethanol, propanol and butanol), may be used in place of some or all of the diacid in the ETPEA-resin forming reaction. The acid halide of the diacid may likewise be employed in place of some or all of the diacid, however such a material is typically much more expensive and difficult to handle compared to the diacid. Likewise, the monoalcohol may be esterified with a volatile acid, e.g., acetic acid, prior to being employed in the ETPEA resin-forming reaction. While such reactive equivalents may be employed in the reaction, their presence may introduce undesired reactive groups into the reaction vessel.

In one embodiment, the equivalents of carboxylic acid may be substantially equal to the combined equivalents of hydroxyl contributed by monoalcohol and polyol, and amine contributed by diamine. In another embodiment, each of the acid and amine equivalents of a resin may be less than 25, such as less than 15, such as less than 10, and further such as less than 5.

When a co-diacid is employed to prepare an ETPEA resin, the co-diacid may contribute up to and including 50% of the equivalents of carboxylic acid present in the reaction mixture. Stated another way, the co-diacid may contribute from 0 to 50 equivalent percent of the carboxylic acid equivalents in the reaction mixture. In one embodiment, the co-diacid may contribute from 0 to 25 equivalent percent, such as from 0 to 10 equivalent percent of the carboxylic acid equivalents in the reaction mixture. In one embodiment, the co-diacid may be chosen from 1,4-cyclohexane dicarboxylic acid, isophthalic acid, adipic acid, azeleic acid, sebacic acid, and dodecandioic acid.

When a co-diamine is employed to prepare an ETPEA resin, the co-diamine present in the reaction mixture may contribute up to and including 50% of the equivalents of amine present in the reaction mixture. Stated another way, the co-diamine may contribute from 0 to 50 equivalent percent of the amine equivalents in the reaction mixture. In one embodiment, the co-diamine may contribute from 0 to 25 equivalent percent, such as from 0 to 10 equivalent percent, of the amine equivalents in the reaction mixture. In another embodiment, the co-diamine may be chosen from 1,6-hexanediamine, xylenediamine, 1,2-propanediamine, 2-methylpentamethylenediamine, and 1,12-dodecanediamine.

The hydroxyl equivalents from polyol may be less than or equal to 50% of the total hydroxyl and amine equivalents contributed by the total of the polyol, monoalcohol and diamine reactants. In another embodiment, the hydroxyl equivalents from polyol may be less than or equal to 40%, such as less than or equal to 30%, and further such as less than or equal to 20%, of the total hydroxyl and amine equivalents contributed by the total of the polyol, monoalcohol and diamine reactants.

The amine equivalents from diamine may equal from 0.3 to 0.75 of the total amine and hydroxyl equivalents provided by diamine, polyol and mono-alcohol. In another aspect, the hydroxyl equivalents from polyol may range from 0.05 to 0.45 of the total amine and hydroxyl equivalents provided by diamine, polyol and mono-alcohol. In another aspect, the hydroxyl equivalents from mono-alcohol may range from 0.20 to 0.45 of the total amine and hydroxyl equivalents provided by diamine, polyol and mono-alcohol.

For example, in one aspect the ETPEA resin may be a resin prepared as described herein where the amine equivalents from diamine may range from 0.30 to 0.75 of the total amine and hydroxyl equivalents provided by diamine, polyol and mono-alcohol; the hydroxyl equivalents from polyol may range from 0.05 to 0.45 of the total amine and hydroxyl equivalents provided by diamine, polyol and mono-alcohol; and the hydroxyl equivalents from mono-alcohol may range from 0.20 to 0.45 of the total amine and hydroxyl equivalents provided by diamine, polyol and monoalcohol. As another example, the ETPEA resin may be a resin prepared by reacting dibasic acid, diamine, polyol and monoalcohol wherein polymerized fatty acid comprises at least 60 equivalent percent of the acid equivalents of the dibasic acid, ethylenediamine comprises at least 75 equivalent percent of the amine equivalents of the amine; and wherein the amine equivalents from diamine may range from 0.30 to 0.75 of the total amine and hydroxyl equivalents provided by diamine, polyol and mono-alcohol; the hydroxyl equivalents from polyol may range from 0.05 to 0.45 of the total amine and hydroxyl equivalents provided by diamine, polyol and mono-alcohol; and the hydroxyl equivalents from mono-alcohol may range from 0.20 to 0.45 of the total amine and hydroxyl equivalents provided by diamine, polyol and mono-alcohol.

In one embodiment, polymerized fatty acid comprises at least 75 equivalent percent, such as at least 90 equivalent percent, of the acid equivalents of the dibasic acid. In another embodiment, polymerized fatty acid comprises at least 75 equivalent percent of the acid equivalents of the dibasic acid, and ethylenediamine comprises at least 75 equivalent percent of the amine equivalents of diamine.

The ETPEA resin can be prepared as described in U.S. Pat. No. 6,552,160, which is herein incorporated by reference.

The ETPEA resin may be, for example, Sylvaclear C 75 V sold by Arizona Chemical.

Amide-Terminated polyamide polymer

The at least one first polymer or second polymer can be an amide-terminated polyamide polymer.

According to one embodiment, the at least one second polymer comprises at least one terminal fatty chain bonded to the polymer skeleton via at least one tertiary amide linking group, and the at least one first polymer comprises at least one terminal fatty chain bonded to the polymer skeleton via at least one ester linking group.

In one embodiment, the tertiary amide-terminated polyamide (ATPA) may be of the formula (IIa): 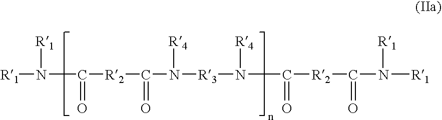
wherein:
n designates a number of repeating units such that terminal amide groups comprise from 10% to 50% of the total amide groups;
R′₁ at each occurrence is independently chosen from a C₁₋₂₂ hydrocarbon group;
R′₂ at each occurrence is independently chosen from a C₂₋₄₂ hydrocarbon group;
R′₃ at each occurrence is independently chosen from an organic group comprising at least two carbon atoms in addition to hydrogen atoms, and optionally comprising one or more atoms chosen from oxygen and nitrogen atoms; and
R′₄ at each occurrence is independently chosen from hydrogen, C₁₋₁₀ alkyl and a direct bond to R′₃ or another R′₄ such that the N atom to which R′₃ and R′₄ are both bonded is part of a heterocyclic structure defined in part by R′₄—N—R′₃.

As may be seen from formula (IIa), the ATPA resins have terminal amide groups of the formula —C(═O)N(R′₁)(R′₁) at both ends of a series of amide groups. These terminal amide groups may be formed from secondary amines (since R′₁ may be an organic group and not hydrogen), and therefore the terminal amide groups in formula (IIa) may be properly referred to as tertiary amide groups. Accordingly, the ATPA resins may be referred to as tertiary amide terminated polyamides.

In some embodiments, R′₁ at each occurrence may be independently chosen from a C₄₋₂₂ hydrocarbon group, R′₂ at each occurrence may be independently chosen from a C₄₋₄₂ hydrocarbon group, and/or R′₃ at each occurrence may be independently chosen from a C₂₋₄₂ hydrocarbon group, where at least 50% of the R′₂ groups comprise from 30 to 42 carbon atoms.

In one embodiment, the resin composition further comprises a diamide of formula (IIa) wherein n=0, such that the ratio of terminal amide groups to the sum of amide groups in the total of the molecules that comprise the resin of formula (IIa) may range from 0.1 to 0.7. In a further embodiment, the resin composition may be at reaction equilibrium.

The letter “n” in formula (IIa) designates the number of repeating units present in a molecule of ATPA, and may be an integer greater than 0. The letter n may be 1, in which case the ATPA comprises equal numbers of terminal amide and non-terminal amide groups, i.e., the terminal amide groups constitute 50% of the total of. the amide groups in the ATPA molecule. In another embodiment, ATPA resins may be of relatively low molecular weight, such that n ranges from 1 to 10, and further from 1 to 5. The terminal amide groups may comprise from about 10% to about 50%, further from 15% to 40%, and further from 20% to 35% of the total of the amide groups. In one embodiment, the ATPA resin comprises a mixture of ATPA molecules of formula (IIa) wherein n may vary. The ATPA resin may have a weight average molecular weight of less than 10,000, such as less than 5,000, but more than 500, such as more than 1,000, when measured by gel permeation chromatography using polystyrene calibration standards.

The R′₁ group in formula (IIa) may be a C₁₋₂₂ hydrocarbon group, such as an alkyl or alkenyl group that comprises at least 1, such as at least 4, and further such as more than 4 carbon atoms. Non-limiting exemplary R′₁ groups comprise 8, 10, 12, 14, 16, 18, 20, or 22 carbon atoms. In one aspect of the present disclosure, R′₁ may be chosen from alkyl groups. In another aspect, alkenyl groups comprising 1-3, such as 1, sites of unsaturation may be chosen for R′₁. The upper range for the number of carbon atoms in the R′₁ group may not be critical, however in one embodiment, the R′₁ group may comprise less than or equal to about 22 carbon atoms. In a further embodiment, the R′₁ group may comprise about 16-22 carbon atoms. The identity of R′₁ at any occurrence is independent of the identity of R′₁ at any other occurrence.

In one embodiment, R′₁ groups may be readily introduced into a molecule of formula (IIa) when one or more secondary monoamines are used as a co-reactant in preparing the ATPA resin. The secondary monoamine comprises the formula HN(R′₁)(R′₁), wherein R′₁ is defined above. In one embodiment, di(hydrogenated tallow) amine may be the secondary monoamine.

The R′₂ group in formula (IIa) may be a hydrocarbon comprising from 2 to 42 carbon atoms, such as from 4 to 42 carbon atoms. In another embodiment, the R′₂ group comprises 30-42 carbon atoms (i.e., is a C₃₀₋₄₂ group). At least 50% of the R′₂ groups in an ATPA resin may comprise from 30 to 42 carbon atoms. Such R′₂ groups may be readily introduced into an ATPA resin when the resin is prepared from polymerized fatty acid, also known as dimer acid.

In one aspect, ATPA resins may comprise at least 50% C₃₀₋₄₂ groups as the R′₂ group, such as at least 75% C₃₀₋₄₂ groups, and further, such as at least 90% C₃₀₋₄₂ groups. One embodiment relates to ATPA resins of formula (IIa) wherein R′₂ may entirely comprise C₃₀₋₄₂ groups.

However, ATPA resins may also comprise R′₂ groups comprising less than 30 carbon atoms. For example, an ATPA resin may comprise one or more R′₂ groups comprising from 4 to 19, such as from 4 to 12, and such as from 4 to 8 carbon atoms. The carbon atoms may be arranged in a linear, branched or cyclic fashion, and unsaturation may be present between any two carbons. Thus, R′₂ may be aliphatic or aromatic. When present, these lower carbon-number R′₂ groups may be formed entirely of carbon and hydrogen, i.e., are hydrocarbon groups. Such lower carbon-number R′₂ groups may comprise less than 50%, such as from 1% to 50%, further such as from 5% to 35%, of the total of the R′₂ groups. The identity of R′₂ at each occurrence is independent of the identity of R′₂ at any other occurrence. Suitable co-diacids are available from, for example, Aldrich (Milwaukee, Wisc.).

The —N(R′₄)—R′₃—N(R′₄)— group in formula (IIa) links two carbonyl (C═O) groups. In one embodiment, all of the R′₄ groups in an ATPA resin are hydrogen, so that R′₃ alone joins the two nitrogen atoms shown in the formula —N(R′₄)—R′₃—N(R′₄)—. In this embodiment, the R′₃ group comprises at least two carbon atoms, and optionally oxygen and/or nitrogen atoms, in addition to any hydrogen atoms that are necessary to complete otherwise unfilled valencies of the carbon, oxygen and nitrogen atoms. In another embodiment, R′₃ may be a hydrocarbon group, comprising from 2 to 36 carbon atoms, such as from 2 to 12 carbon atoms, and further such as from 2 to 8 carbon atoms. These carbon atoms may be arranged in a linear, branched or cyclic fashion, and unsaturation may be present between any two of the carbon atoms. Thus, R′₃ may be aliphatic or aromatic. The identities of R′₃ and R′₄ at each occurrence are independent of their identities at any other occurrence.

The R′₃ groups may comprise at least one oxygen and/or nitrogen in addition to carbon and hydrogen atoms. In one aspect, an R′₃ group comprising at least one oxygen atom may be a polyalkylene oxide, i.e., a group comprising alternating alkylene groups and oxygen atoms. For example, the oxygenation in a R′₃ group may be present as an ether group. Representative polyalkylene oxides include, without limitation, polyethylene oxide, polypropylene oxide and copolymers (either random, alternating or block) of ethylene oxide and propylene oxide. Such oxygenated R′₃ groups may be readily introduced into an ATPA resin through use of JEFFAMINE™ diamines (Huntsman Chemical, Inc., Houston, Tex.). These materials are available in a wide range of molecular weights, where any molecular weight diamine may be used in the preparation of the ATPA resins. While some of the R′₃ groups may comprise oxygen atoms (at least about 1%), in one embodiment, less than 50% of the R′₃ groups comprise oxygen atoms, such as less than 20% of the R′₃ groups comprise oxygen atoms. The presence of R′₃ groups comprising at least one oxygen atom may lower the softening point of the ATPA resin.

When present, the nitrogen atoms in an R′₃ group may be present as secondary or tertiary amines. In one embodiment, a typical nitrogenated R′₃ group comprising secondary amine groups may be a polyalkylene amine, i.e., a group comprising alternating alkylene groups and amine groups, which may be referred to as a polyalkylene polyamine. In another embodiment, the alkylene group may be a lower alkylene group; non-limiting examples include methylene, ethylene, (i.e., —CH₂—CH₂—), and propylene. A polyalkylene amine may be represented by the formula —NH—(CH₂—CH₂—NH)ₘ—CH₂—CH₂—NH—, wherein m is an integer from 1 to 5.

However, the nitrogen atoms in the nitrogenated R′₃ group may alternatively (or additionally) be present as tertiary nitrogen atoms. In one embodiment, the nitrogen atoms may be present in a heterocycle of the formula: 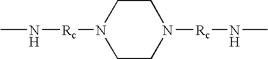
wherein R_{c} is a C₁₋₃ alkylene group.

In the above-described nitrogen-containing R′₃ groups, R′₄ was hydrogen. However, R′₄ is not limited to hydrogen. In fact, R′₄ may be a C₁₋₁₀ alkyl group, such as a C₁₋₅ alkyl group, and further such as a C₁₋₃ alkyl group. In one aspect, R′₃ and R′₄, or two R′₄ groups, may together form a heterocyclic structure, for example, a piperazine structure such as

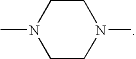

In this case, the two R′₄ groups may be seen as joining together to form an ethylene bridge between the two nitrogen atoms, while R′₃ is also an ethylene bridge. Additional suitable diamines may be available from, for example, Aldrich (Milwaukee, Wisc.).

The ATPA resin may be, for example, Sylvaclear A 200 V sold by Arizona Chemical.

The ATPA resin can be prepared as described in U.S. Pat. No. 6,503,522, which is herein incorporated by reference.

Ether-Terminated poly(ether-amide)

The at least one first polymer and/or second polymer comprises at least one terminal fatty chain bonded to the polymer skeleton via at least one ether linking group or polyether linking group.

According to one embodiment, the at least one second polymer comprises at least one terminal fatty chain bonded to the polymer skeleton via at least one ether linking group or polyether linking group, and the at least one first polymer comprises at least one terminal fatty chain bonded to the polymer skeleton via at least one ester linking group.

The polymer can be a block copolymer of the ether terminated poly(amide-ether) type.

The polymer can also be chosen from polyamide polymers of formula (II) wherein -L- is a group of formula: 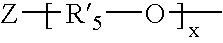
wherein
R′₅ is chosen from C₂-C₆ hydrocarbon diradicals;
Z is chosen from O and NH; and
x is an integer ranging from 2 to 100.

In one embodiment, the polymer may be chosen from polyamide polymers of formula (IIb): 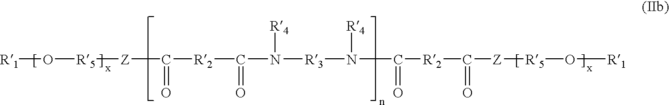
wherein
R′₁, which may be identical or different, are each independently chosen from C₁-C₂₂ alkyl and C₁-C₂₂ alkylene radicals;
Z is chosen from O and NH;
x is an integer from 2 to 100;
R′₂, which may be identical or different, are each independently chosen from C₂ to C₅₂ hydrocarbon diradicals, wherein at least 50% of the R′₂ comprise at least 34 carbon atoms;
R′₃, which may be identical or different, are each independently chosen from C₂-C₃₆ hydrocarbon diradicals and C₄-C₁₀₀ polyether diradicals;
R′₄, which may be identical or different, are each independently chosen from hydrogen atoms, C₁ to C₁₀ alkyl groups and a direct bond to at least one group chosen from R′₃ and another R′₄ such that when a direct bond to at least one group from another R₄ is chosen, the nitrogen atom to which both R′₃ and R′₄ are bonded forms part of a heterocyclic structure defined in part by R′₄—N—R′₃, with the proviso that at least 50% of all R′₄ are chosen from hydrogen atoms;
R′₅ is chosen from C₂-C₆ hydrocarbon diradicals; and
n is an integer from 1 to 10.

In one embodiment, R′₅ may be a C₂ hydrocarbon diradical, and at least 80% of the R′₂ diradicals may comprise at least 34 carbon atoms. In another embodiment, Z may be NH.

In formula (IIb), a hydrocarbon group comprises only carbon and hydrogen atoms. For example, hydrocarbon groups may be formed from one or more aliphatic and aromatic moieties. Aliphatic moieties useful herein include, but are not limited to, alkyl, alkylene, alkenyl, alkenylene, alkynyl, alkylnylene, cycloalkyl, cycloalkylene, cycloalkenyl, cycloalkenylene, cycloalkynyl, and cycloalkynylene moieties. Aromatic moieties may also be referred to herein as aryl groups. The hydrocarbon group may be referred to herein as R′₁.

As used herein, alkyl, alkenyl, alkynyl,cycloalkyl, cycloalkenyl, and cycloalkynyl refer to monovalent radicals, while alkylene, alkenylene, alkynylene, cycloalkylene, cycloalkenylene, and cycloalkynylene refer to polyvalent radicals. As used herein, alkyl, alkylene, cycloalkyl, and cycloalkylene refer to saturated radicals, while alkenyl, alkenylene, alkynyl, alkylnylene, cycloalkenyl, cycloalkenylene, cycloalkynyl, and cycloalkynylene refer to unsaturated radicals. The alkyl, alkylene, alkenyl, alkenylene, alkynyl, and alkylnylene moieties may be straight-chained or branched. The cycloalkyl, cycloalkenyl, cycloalkynyl, cycloalkylene, cycloalkenylene and cycloalkynylene moieties may be monocyclic or polycyclic, where a polycyclic moiety may be, for example, bicyclic or tricyclic.

Non-limiting exemplary alkyl moieties include methyl, ethyl, propyl, hexyl, and 2-ethylhexyl. Non-limiting exemplary alkylene moieties include methylene (—CH₂—), methylidene (═CH₂), and ethylene (—CH₂—CH₂—). Non-limiting exemplary cycloalkyl groups include cyclohexyl and norbornyl.

Aromatic moieties useful herein may be monocyclic or polycyclic. A non-limiting exemplary monocyclic aryl group may be phenyl, while exemplary polycyclic aryl groups include, but are not limited to, naphthyl and fulverenyl. The aromatic moiety may be monovalent, e.g., phenyl, or polyvalent, e.g., phenylene.

In one embodiment, the hydrocarbon group may comprise a combination of aromatic and aliphatic groups. Non-limiting examples include benzyl (phenyl-CH₂—, an arylalkylene group), tolyl (CH₃-phenylene-, an alkylarylene group), and xylyl ((CH₃)₂phenylene-, a dialkylarylene group). In another embodiment, the hydrocarbon group may comprise a combination of two or more aromatic groups, e.g., biphenyl (phenyl-phenylene-, an arylarylene group).

In one embodiment, the R″₁ group comprises 1 to 32 carbon atoms. In one embodiment, the R″₁ alkyl group comprises 1 to 12 carbon atoms. In another embodiment, the R″₁ group may be an alkyl group. In another embodiment, the R″₁ alkyl group may be straight-chained. In yet another embodiment, the R″₁ alkyl group may be branched.

The block copolymer of formula (lib) may comprise at least two polyether blocks. A polyether block comprises a plurality of ether groups, i.e., groups of the formula —C—O—C—. In one aspect, R′₃ may be a polyether.

In one embodiment, a polyether block may comprise the repeating formula —O—R″₂—, where R″₂ may be a hydrocarbon group. In one aspect, R″₂ may be an alkylene group. The alkylene group R″₂ may be aliphatic (saturated and/or unsaturated) or aromatic, straight-chained and/or branched, independently at each occurrence in the polyether block. In one aspect, R″₂ may comprise from 1 to 6 carbon atoms at each occurrence in the polyether block, while in another aspect, R″₂ comprises from 2 to 4 carbon atoms at each occurrence. In one aspect, R″₂ may comprise the formula —CH₂—CH(R″₂ₐ)—, wherein R″₂ₐ may be chosen from hydrogen, methyl and ethyl.

In one aspect, the polyether component of the block copolymer may have a molecular weight (number or weight average) of less than 10,000. In another aspect, the molecular weight may range from 100 to 4,000.

The block copolymer of formula (IIb) may comprise a polyamide block. The polyamide block may comprise a plurality of amide groups, i.e., groups of the formula —NH—C(═O)— and/or —C(═O)—NH—. In the polyamide block, two or more amide groups may be separated by hydrocarbon groups, e.g., alkylene groups and/or polyether groups.

In one aspect, the polyamide block comprises —C(O)—R″₃—C(O)— moieties wherein R″₃ is a hydrocarbon group. In one aspect, the polyamide block includes R″₃ groups comprising at least 30 carbon atoms. In one aspect, the polyamide block includes R″₃ groups comprising from 30 to 42 carbon atoms.

In one aspect, the polyamide block includes R″₃ groups that may be formed from fatty acid polymerization.

In one aspect, the block copolymers may be of formula (IIb), wherein each of the C(═O) groups may be bonded to a C₃₄ hydrocarbon group, i.e., the block copolymer may be formed from dimer acid as the exclusive polyacid reactant. However, in another aspect, the polyamide block includes both C₃₄ and “co-diacid”-derived R″₃groups. Thus, the polyamide block may be formed by reacting both dimer acid and co-diacid with a diamine.

As used herein, a co-diacid refers to a compound of formula HOOC—R″₃—COOH, where R″₃ is not a C₃₄ hydrocarbon group as defined above. In one aspect, the polyamide block in copolymers of formula (IIb) includes R″₃ groups comprising from 2 to 32 carbons, which may be referred to herein as co-diacid R″₃ groups. Co-diacid R″₃ groups useful herein include, but are not limited to, ethylene (from, e.g., succinic acid) and n-butylene (from, e.g., adipic acid).

In one aspect, the C₃₄ R″₃ groups may comprise at least 50 mol % of the total of the R₃ groups. In other aspects, the C₃₄ R″₃ groups may comprise at least 60 mol %, such as at least 70 mol %, such as at least 80 mol %, such as at least 90 mol %, and further such as at least 95 mol % of the R″₃ groups. Stated another way, dimer acid may comprise at least 50% of the diacid equivalents, such as at least 60%, such as at least 70%, such as at least 80%, such as at least 90%, and further such as at least 95% of the diacid equivalents in the polyamide block of the copolymer of formula (IIb).

In one aspect, the polyamide block may comprise —NH—R″₄—NH— moieties wherein R″₄ is a hydrocarbon group. In one aspect, the R″₄ hydrocarbon groups may comprise from 1 to 20 carbons. In another aspect, the polyamide block includes R″₄ groups comprising from 1 to 10 carbons. In yet another aspect, the R″₄ group may be an alkylene group, such as a straight-chained alkylene group. In one aspect, the polyamide block includes R″₄ groups comprising 2 carbons, while in another aspect, at least 50% of the R″₄ groups may comprise 2 carbons, while in a further aspect, all of the R″₄ groups may comprise 2 carbons.

In one aspect, the polyamide block may comprise —NH—R″₄—NH— moieties wherein R″₄ may be a polyether group. As used herein, a polyether block comprises a plurality of ether groups, i.e., groups of the formula —C—O—C—. In other words, a polyether block may comprise the repeating formula —O—R″₂— where R″₂ is a hydrocarbon group. In one aspect, R″₂ may be an alkylene group. The alkylene group R″₂ may be aliphatic (saturated and/or unsaturated) or aromatic, straight chain and/or branched, independently at each occurrence in the polyether block. In one aspect, R″₂ may comprise from 1 to 6 carbon atoms at each occurrence in the polyether block, while in another aspect R″₂ has from 2 to 4 carbons at each occurrence. In one aspect, R″₂ may comprise the formula —CH₂—CH(R″₂ₐ)—, wherein R″₂ₐ is chosen from hydrogen, methyl and ethyl.

In one aspect, the polyether component of the R″₄ portion of the block copolymer may have a molecular weight (number or weight average) of less than 10,000. In another aspect, the molecular weight may range from 100 to 4,000.

Compounds of the formula H₂N—R″₄—NH₂ are commonly known as diamines, and may be available from a large number of vendors. Compounds of the formula HOOC—R″₃—COOH are commonly known as diacids, or dibasic acids.

In formula (IIb), the bond “—” between hydrocarbon and polyether represents a C—O bond where the carbon is contributed by the hydrocarbon and the oxygen is contributed by the polyether.

In formula (IIb), in one aspect, the bond between polyether and polyamide is C—NH—C(═O)—C where C—NH may be seen as being contributed by the polyether and C(═O)—C may be seen as being contributed by the terminal acid group of a polyamide. Block copolymers according to this aspect may be formed by, for example, reacting an amino and hydrocarbon-terminated polyether of the formula R″₁—(O—R″₂—)NH₂ with a carboxylic acid-terminated polyamide of the formula HOOC—NH−R″₄—NH— so as to form R″₁—(O—R″₂—)NH—C(═O)—NH—R″₄—NH—. Thus, an amide group may be present as the link between polyether and polyamide in formula (IIb).

In formula (IIb), in one aspect, the bond between polyether and polyamide may be C—C(═O)—NH—C where C—C(═O) may be seen as being contributed by the polyether and NH—C may be seen as being contributed by the terminal amine group of a polyamide. Block copolymers according to this aspect may be formed by, for example, reacting a carboxylic acid and hydrocarbon-terminated polyether of the formula R″₁—(O—R″₂—)COOH with an amine terminated polyamide of the formula H₂N—R″₄-NH—C(═O)—R″₃— so as to form R″“—(O—R″₂—)—C(═O)NH—R″₄—NH—C(═O)—R″₃. Thus, once again, an amide group may be present as the link between polyether and polyamide in formula (IIb).

In formula (IIb), in one aspect, the bond between polyether and polyamide is C—O—C(═O)—C where C—O may be seen as being contributed by the polyether and C(═O)—C may be seen as being contributed by the terminal acid group of a polyamide. Block copolymers according to this aspect may be formed by, for example, reacting a hydroxyl and hydrocarbon-terminated polyether of the formula R″₁—(O—R″₂)OH with a carboxylic acid terminated polyamide of the formula HOOC—NH—R″₄—NH— so as to form R″₁—(O—R″₂—)—O—C(═O)—NH—R″₄—NH. Thus, an ester group may be present as the link between polyether and polyamide in formula (IIb).

In one aspect, the hydrocarbon-terminated polyether-polyamide block copolymers may have a softening point of 50 to 150° C. (as determined by Ring and Ball, or Mettler methods). In another aspect, the softening point may range from 75 to 125° C., while in a further aspect, the softening point may range from 75 to 100° C., while in another aspect, the softening point may range from 80 to 120° C.

In one aspect, the hydrocarbon-terminated polyether-polyamide block copolymers, may have a weight or number average molecular weight ranging from 2,000 to 30,000. The molecular weight may be measured by preparing a solution of the copolymer or composition in a suitable solvent, e.g., tetrahydrofuran (THF), identifying the retention time of the copolymer by gel permeation chromatography, and comparing that retention time to the retention times of solutions of polystyrene having known molecular weight characterizations. In one aspect, the copolymers may have a weight or number average molecular weight of greater than 1,000.

In one aspect, the ether-terminated polyether-polyamide block copolymers, may have a viscosity, at 160° C., of less than 5,000 centipoise (cPs, or cps), such as less than 4,000 cPs, such as less than 3,000 cPs, such as less than 2,000 cPs, and further such as less than 1,000 cPs. In one embodiment, the copolymers have a melt viscosity, as measured on the neat copolymer or composition at 160° C., of more than 50 cPs, such as more than 500 cPs.

The ether-terminated polyether-polyamide resin can be prepared as described in U.S. Pat. No. 6,399,713, which is herein incorporated by reference.

Second polymer

In one aspect of the present disclosure, the at least one second polymer of the composition comprises
a) a polymer skeleton which comprises at least one hydrocarbon-based repeating unit comprising at least one heteroatom, and
b) at least one of:
   at least one terminal fatty chain chosen from alkyl chains and alkenyl chains, wherein the at least one terminal fatty chain is bonded to the polymer skeleton via at least one linking group; and
   at least one pendant fatty chain chosen from alkyl chains and alkenyl chains, wherein the at least one pendant fatty chain is bonded to the polymer skeleton via at least one linking group.

In one embodiment, the at least one second polymer may be a structuring polymer for the liquid fatty phase, such as a polymer with a polymer skeleton comprising at least one polyamide block. In another embodiment, the polymer skeleton may be chosen from a polyamide skeleton, a polyamide-polyester block skeleton, and a polyamide-polyether skeleton.

The at least one second polymer may have a softening point of greater than 50° C., such as from 65° C. to 190° C., and less than 150° C., such as from 70° C. to 130° C., and even further such as from 80° C. to 105° C. The softening point can be measured by a well known method as “Differential Scanning Calorimetry” (i.e., DSC method) with a temperature rise of 5 to 10° C./min or Ring and Ball method. In one aspect, the polymer may be a non-waxy polymer.

According to one embodiment, the at least one second polymer comprises at least one terminal fatty chain bonded to the polymer skeleton via at least one linking group chosen from single bonds and urea, urethane, thiourea, thiourethane, thioether, thioester, ether, amide, tertiary amide or secondary amide groups.

In another embodiment, the at least one second polymer may be a polyamide polymer comprising at least one terminal fatty chain bonded to the polymer skeleton via at least one tertiary amide linking group, wherein the first polymer is an ester terminated polyamide as described above.

In one embodiment, the at least one second polymer comprises at least one terminal fatty chain bonded to the polymer skeleton via at least one ether group or polyether group.

The at least one second polymer may be present in the composition in an amount ranging from 0.5% to 80% by weight relative to the total weight of the composition, such as ranging from 2% to 60%, such as from 5% to 40%, such as from 5% to 25% and further such as from 5% to 15%.

Hardness and Stability of the Composition

The concentrations of the at least one first polymer and of the at least one second polymer may be chosen according to the desired hardness and desired stability of the compositions and according to the specific application envisaged. The respective concentrations of the at least one first polymer and of the at least one second polymer can be such that a disintegrable solid which does not flow under its own weight may be obtained.

Depending on the intended application, such as a stick, the hardness of the composition may also be considered. The hardness of a composition may, for example, be expressed in units of gram force (gf). The composition may, for example, have a hardness ranging from 20 gf to 2000 gf, such as from 20 gf to 900 gf, and further such as from 20 gf to 600 gf.

This hardness may be measured in two ways.

The first test for hardness includes a method of penetrating a probe into the composition and in one aspect, using a texture analyzer (for example, TA-XT2i from Rhéo) equipped with an ebonite cylinder of height 25 mm and diameter 8 mm. This hardness measurement may be carried out at 20° C. at the center of 5 samples of the composition. The cylinder may be introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 gf.

The second test for hardness includes the “cheese wire” method OSI which involves cutting a sample of composition that is 8.1 mm, such as 12.7 mm in diameter and measuring its hardness at 20° C. using a DFGHS 2 tensile testing machine from Indelco-Chatillon Co. at a speed of 100 mm/minute. The hardness value from this method is expressed in grams force, as the shear force required to cut a stick under the above conditions. The hardness of compositions which may be in stick form may, for example, range from 30 gf to 300 gf, such as from 30 gf to 250 gf, and further such as from 30 gf to 200 gf.

In one aspect, the hardness of the composition may be such that the compositions are self-supporting and can easily disintegrate to form a satisfactory deposit on a keratinous material. In addition, this hardness may impart good impact strength to the compositions which may be molded or cast, for example, in stick or dish form.

The ratio of the first polymer and second polymer may range from 1/10 and 10/1, such as from 1/5 and 5/1, such as from 1/2 to 4/1, or from 4/1 to 5/1, and further such as 1/1 or 3/1.

The skilled artisan may choose to evaluate a composition using at least one of the tests for hardness outlined above based on the application envisaged and the hardness desired. Obtaining an acceptable hardness value, in view of the intended application, from at least one of these hardness tests may comprise an aspect of the present disclosure.

According to one embodiment, the compositions in stick form may also possess the properties of deformable, flexible elastic solids and may also have noteworthy elastic softness upon application to a keratinous material.

Amphiphilic Compound

In one embodiment, the composition may comprise at least one amphiphilic liquid component at ambient temperature, with a hydrophilic/lipophilic balance (HLB) lower than 12, such as from 1 to 7, such as from 1 to 5, and further such as from 3 to 5.

The amphiphilic components may include a lipophilic part linked to a polar part, the lipophilic part comprising a carbon chain, comprising at least 8 carbon atoms, such as from 18 to 32 carbon atoms, and further such as from 18 to 28 carbon atoms. In one embodiment, the polar part of at least one amphiphilic component may be the reaction residue of a component chosen from among the alcohols and polyols comprising from 1 to 12 hydroxyl groups, the polyoxalkylenes comprising at least 2 oxyalkenated moieties and comprising from 0 to 20 oxypropylenated moieties and/or 0 to 20 oxyethylenated moieties. In one aspect, the amphiphilic component may be an ester chosen from the reaction products of hydroxystearates, oleates, or isostearates with glycerol, sorbitan, methylglucose or the fatty alcohols in the C₁₂ to C₂₆ range, such as octyidodecanol, and mixtures of these. In another aspect, these esters may be chosen from the monoesters and the mono- and di-ester.

The amount of amphiphilic component may be chosen according to the desired hardness of the composition and according to the intended application.

Liquid Fatty Phase

The at least one liquid fatty phase may comprise at least one oil. In one embodiment, the at least one oil has an affinity with the first polymer and/or the second polymer. The at least one oil, for example, may be chosen from polar oils and apolar oils, including hydrocarbon-based liquid oils and oily liquids at room temperature.

In one embodiment, the composition comprises at least one structuring polymer and at least one polar oil. The structuring polymer may be chosen from the first polymer, the second polymer and mixtures thereof.

As used herein, the expression “hydrocarbon-based oil” refers to an oil comprising carbon and hydrogen atoms, optionally with at least one group chosen from hydroxyl, ester, carboxyl, or ether groups.

For example, the at least one polar oil may be chosen from:
hydrocarbon-based plant oils with a high content of triglycerides comprising fatty acid esters of glycerol wherein the fatty acids comprise chains having from 4 to 24 carbon atoms, these chains possibly being chosen from linear and branched, and saturated and unsaturated chains; these oils may be chosen from, for example, wheat germ oil, corn oil, sunflower oil, karite butter, castor oil, sweet almond oil, macadamia oil, apricot oil, soybean oil, cotton oil, alfalfa oil, poppy oil, pumpkin oil, sesame oil, marrow oil, rapeseed oil, avocado oil, hazelnut oil, grape seed oil, blackcurrant seed oil, evening primrose oil, millet oil, barley oil, quinoa oil, olive oil, rye oil, safflower oil, candlenut oil, passion flower oil and musk rose oil; or alternatively caprylic/capric acid triglycerides such as those sold by Stearineries Dubois, or those sold under the names Miglyol 810, 812 and 818 by Dynamit Nobel;
synthetic oils or esters of formula R₅COOR₆, wherein R₅ is chosen from linear and branched fatty acid residues comprising from 1 to 40 carbon atoms, and R₆ may be chosen from, for example, alkyl groups comprising from 1 to 40 carbon atoms, with the proviso that R₅+R_{6≧10;} non-limiting examples include purcellin oil (cetostearyl octanoate), isononyl isononanoate, C₁₂-C₁₅ alkyl benzoates, isopropyl myristate, 2-ethylhexyl palmitate, isostearyl isostearate, alkyl or polyalkyl octanoates, decanoates or ricinoleates; hydroxylated esters such as isostearyl lactate and diisostearyl malate; and pentaerythritol esters;
synthetic ethers comprising from 10 to 40 carbon atoms;
C₈ to C₂₆ fatty alcohols such as oleyl alcohol; and
C₈ to C₂₆ fatty acids such as oleic acid, linolenic acid and linoleic acid.

The at least one apolar oil may be chosen from, for example, silicone oils chosen from volatile and non-volatile, linear and cyclic polydimethylsiloxanes (PDMSs) that are liquid at room temperature; polydimethylsiloxanes comprising alkyl or alkoxy groups, wherein each alkyl or alkoxy group may be independently chosen from being pendant and being at the end of the silicone chain, and wherein the groups each comprise from 2 to 24 carbon atoms; phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; hydrocarbons chosen from linear and branched, volatile and non-volatile hydrocarbons of synthetic and mineral origin, such as volatile liquid paraffins (such as isoparaffins and isododecane) or non-volatile liquid paraffins and derivatives thereof; liquid petrolatum, liquid lanolin, polydecenes, hydrogenated polyisobutene such as hydrogenated polybutene, e.g., Parleam® from Nippon Oil Fats and squalane; and mixtures thereof. The structured oils, for example those structured with polyamides such as those of formula (III) or the polyurethanes or polyureas or polyurea-urethanes, may be, in one embodiment, apolar oils, such as an oil or a mixture of hydrocarbon oils chosen from those of mineral and synthetic origin, hydrocarbons such as alkanes such as Parleam® oil, isoparaffins including isododecane, and squalane, and mixtures thereof. These oils may, in one embodiment, be combined with at least one phenylsilicone oil.

The liquid fatty phase, in one embodiment, comprises at least one non-volatile oil chosen from, for example, hydrocarbon-based oils of mineral, plant and synthetic origin, synthetic esters or ethers, silicone oils and mixtures thereof.

In one embodiment, the total liquid fatty phase may be present, for example, in an amount ranging from 1% to 99% by weight relative to the total weight of the composition; further non-limiting examples include ranges of 5% to 99%, 5% to 95.5%, 10% to 80%, and 20% to 75%.

As used herein, the expression “volatile solvent or oil” refers to any non-aqueous medium capable of evaporating on contact with the skin or the lips in less than one hour at room temperature and atmospheric pressure. An aspect of the present disclosure includes one or more volatile solvents chosen from organic solvents, such as volatile cosmetic oils that are liquid at room temperature and have a non-zero vapor pressure, at room temperature and atmospheric pressure, ranging from 10⁻² to 300 mm Hg (1.33 to 40,000 Pa), such as greater than 0.03 mmHg (4 Pa), and further such as greater than 0.3 mmHg (40 Pa). The expression “non-volatile oil” as used herein refers to an oil which remains on the skin or the lips at room temperature and atmospheric pressure for at least several hours, such as those having a vapor pressure of less than 10⁻² mmHg (1.33 Pa).

In one embodiment, these volatile solvents may facilitate the staying power or long wearing properties of the composition on the skin, the lips or superficial body growths such as nails and keratinous fibers. The solvents can be chosen from hydrocarbon-based solvents, silicone solvents optionally comprising alkyl or alkoxy groups that are pendant or at the end of a silicone chain, and a mixture of these solvents.

The volatile oil(s), in one embodiment, may be present in an amount ranging from 0% to 95.5% relative to the total weight of the composition, such as from 2% to 75% or, for example, from 10% to 45%. This amount may be adapted by a person skilled in the art according to the desired staying power or long wearing properties.

In one embodiment, the compositions may be free of volatile oil.

Coloring Agent

In one aspect, the composition may be in the form of a tinted or non-tinted care composition for keratin materials such as the skin, the lips and superficial body growths. The tinted or non-tinted composition can be used, for example, as a care base for the skin, superficial body growths or the lips. Non-limiting examples include lip balms for protecting the lips against cold and/or sunlight and/or wind, and care cream for the skin (body and face).

In another aspect, the compositions may be also in the form of colored make-up products for the skin, such as foundations, eyeshadows, concealers, eyeliners, make-up for the body, make-up for the lips such as lipglosses or lipsticks, make-up for eyelashes, for example in a form of mascara cakes, or for the eyebrows, for example in the form of pencils.

In one embodiment, the composition may also comprise at least one coloring agent chosen from pigments and dyes. As used herein, pigments refer to colored solid particles at 25° C. that are not soluble in the liquid fatty phase. Pigments may include nacreous pigments (i.e., nacres), and pearling agents.

The at least one coloring agent may be chosen, for example, in order to obtain make-up compositions which give good coverage, in other words, which do not leave a significant amount of the at least one keratin material to which it is applied showing through. The pigments may also reduce the sticky feel of the compositions, unlike soluble dyes.

Representative liposoluble dyes which may be used include, but are not limited to, Sudan red, DC Red 17, DC Green 6, β-carotene, soybean oil, Sudan brown, DC Yellow 11, DC Violet 2, DC Orange 5, annatto, and quinoline yellow. The liposoluble dyes, when present, may have a concentration ranging up to 20% by weight of the total weight of the composition, such as from 0.1% to 6%.

In one aspect, the pigments may be chosen from white, colored, mineral, organic, coated and uncoated pigments. Representative examples of mineral pigments include, but are not limited to, titanium dioxide, which may be optionally surface-treated, zirconium oxide, zinc oxide, cerium oxide, iron oxides, chromium oxides, manganese violet, ultramarine blue, chromium hydrate and ferric blue. Representative examples of organic pigments include, but are not limited to, carbon black, pigments of D & C type, and lakes based on cochineal carmine, barium, strontium, calcium and aluminum. If present, the pigments may have a concentration ranging up to 40% by weight of the total weight of the composition, and for example up to 50%, such as from 1% to 35%, and further such as from 2% to 25%. In one embodiment comprising a face powder product, the pigments, including nacreous pigments, may, for example, represent up to 90% by weight of the composition.

In one aspect, the nacreous pigments (or nacres) may be chosen from white nacreous pigments such as mica coated with titanium or with bismuth oxychloride; colored nacreous pigments such as titanium mica with iron oxides, titanium mica with ferric blue or chromium oxide, and titanium mica with an organic pigment chosen from those mentioned above; and nacreous pigments based on bismuth oxychloride. The nacres, if present, may have a concentration ranging up to 30% by weight of the total weight of the composition, such as from 0.1% to 20%.

In one embodiment, the coloring agent may be a pigment (nacreous or non-nacreous).

In one embodiment, the compositions may be anhydrous. In another embodiment, the at least one liquid fatty phase of the composition may further comprise a dispersion of lipid vesicles. The composition may also, for example, be in the form of a fluid anhydrous gel, a rigid anhydrous gel, a fluid simple emulsion, a fluid multiple emulsion, a rigid simple emulsion or a rigid multiple emulsion. The simple emulsion or multiple emulsion may comprise a continuous phase chosen from an aqueous phase optionally comprising dispersed lipid vesicles, and a fatty phase optionally comprising dispersed lipid vesicles. In one embodiment, the composition comprises a continuous oily phase or fatty phase and may be an anhydrous composition, for example, in a stick or dish form. An anhydrous composition may be one that has less than 10% water by weight, such as, for example, less than 5% by weight, such as less than 3% by weight, and further such as less than 1% by weight relative to the total weight of the composition.

In one aspect, the composition may be manufactured by one of ordinary skill in the art. For example, the composition may be manufactured by a process which comprises heating the at least one first polymer at least to its softening point, adding the at least one second polymer and any suitable additives, if present, to the at least one first polymer, followed by mixing the composition. The resultant homogeneous mixture may then be cast or poured in a suitable mold such as a lipstick mold, foundation mold, or deodorant mold or cast directly into the packaging articles such as a case or a dish.

A further embodiment includes a skin, lip, or keratinous fiber care or make-up composition comprising a composition as described above.

Additionally, an aspect of the present disclosure relates to a method for care or make up of a keratin material chosen from lips, skin, and keratinous fibers, comprising applying to the skin, lips, or keratinous fibers a composition comprising at least one liquid fatty phase, at least one first polymer comprising a polymer skeleton comprising at least one hydrocarbon-based repeating unit comprising at least one heteroatom and at least one second polymer.

An aspect of the present disclosure includes a cosmetic process for caring for, making up or treating a keratin material, such as that of a human being, and further such as human skin, lips, hair, eyebrows, nails, and eyelashes, comprising the application to a keratin material of a cosmetic composition.

The invention will be illustrated by, but is not intended to be limited to, the following examples. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements. The amounts are given in a percentage by mass.

### EXAMPLE 1

### Clear Lipstick

| TRADE NAME | CTFA NAME | Weight % |
|---|---|---|
| Phase A | | |
| Demol DGDIS | Polyglyceryl-2 diisostearate | 25.0 |
| Dragoxat EH | Octyl octanoate | 20.0 |
| Finsolv TN | C₁₂₋₁₅ alkyl benzoate | 9.0 |
| Polysynlane V | Hydrogenated polyisobutene | 10.0 |
| Eutanol G | Octyl dodecanol | 9.95 |
| DC 556 | Phenyl trimethicone | 5.0 |
| GP-1 | Dibutyl lauroyl glutamide | 1.0 |
| Phase B | | |
| | BHT | 0.05 |
| Sylvaclear A200V | Amide-terminated polyamide | 10.0 |
| Uniclear 100VG | Polyamide resin | 10.0 |

The composition can be prepared as follows:
Phase A is introduced into a heating vessel at a temperature of about 110° C. with mixing until GP-1 completely dissolved. Then Phase B is introduced into the vessel at the same temperature. The mixing and heating can be then continued to obtain a transparent homogeneous liquid. The composition is then cast in a mold.

The composition has good stability: there is no syneresis (also called exudation) at room temperature, at 45° C. and at 50° C., both at one month and at eight weeks.

### EXAMPLE 2

### Clear Lipstick with Color

| TRADE NAME | CTFA NAME | Weight % |
|---|---|---|
| Phase A | | |
| Demol DGDIS | Polyglyceryl-2 diisostearate | 25.0 |
| Dragoxat EH | Octyl octanoate | 24.0 |
| Finsolv TN | C₁₂₋₁₅ alkyl benzoate | 9.0 |
| Polysynlane V | Hydrogenated polyisobutene | 10.0 |
| Eutanol G | Octyl dodecanol | 5.95 |
| DC 556 | Phenyl trimethicone | 5.0 |
| Phase B | | |
| | BHT | 0.05 |
| Sylvaclear C75V | Ester-Amide-Modified Polyamide resin | 5.0 |
| Uniclear 100VG | Ester terminated Polyamide resin | 15.0 |
| Phase C | | |
| | Pigment grind | 1.0 |

The composition can be prepared as follows:
Phase A is introduced into a heating vessel at a temperature of about 110° C. with mixing until GP-1 completely dissolved. Then Phase B is introduced into the vessel at the same temperature. The mixing and heating can be then continued to obtain a transparent homogeneous liquid. The ground pigment material (phase C) is then introduced into the mixture with mixing until the mixture is uniform. The composition is then cast in a mold.)

The composition has good stability in that there is no exudation (or syneresis) at room temperature, at 45° C., and at 50° C., both at one month and at eight weeks.

1. A composition comprising
i) at least one liquid fatty phase,
ii) at least one first polymer comprising
a) a polymer skeleton which comprises at least one hydrocarbon-based repeating unit comprising at least one heteroatom, and
b) at least one of:
at least one terminal fatty chain chosen from alkyl chains and alkenyl chains, wherein the at least one terminal fatty chain is bonded to the polymer skeleton via at least one ester linking group; and
at least one pendant fatty chain chosen from alkyl chains and alkenyl chains, wherein the at least one pendant fatty chain is bonded to the polymer skeleton via at least one ester linking group, and
iii) at least one second polymer, different from the first polymer, comprising
a) a polymer skeleton which comprises at least one hydrocarbon-based repeating unit comprising at least one heteroatom, and
b) at least one of:
at least one terminal fatty chain chosen from alkyl chains and alkenyl chains, wherein the at least one terminal fatty chain is bonded to the polymer skeleton via at least one amide linking group; and
at least one pendant fatty chain chosen from alkyl chains and alkenyl chains, wherein the at least one pendant fatty chain is bonded to the polymer skeleton via at least one amide linking group,
wherein the second polymer does not comprise an ester linking group.

2. A composition according to claim 1, wherein the at least one first polymer further comprises at least one of:
at least one terminal fatty chain chosen from alkyl chains and alkenyl chains, wherein the at least one terminal fatty chain is bonded to the polymer skeleton via at least one linking group; and
at least one pendant fatty chain chosen from alkyl chains and alkenyl chains,
wherein the at least one pendant fatty chain is bonded to the polymer skeleton via at least one linking group.

3. A cosmetic composition comprising
i) at least one liquid fatty phase,
ii) at least one first polymer comprising
a) a polymer skeleton which comprises at least one hydrocarbon-based repeating unit comprising at least one heteroatom, and
b) at least one of:
at least one terminal fatty chain chosen from alkyl chains and alkenyl chains, wherein the at least one terminal fatty chain is bonded to the polymer skeleton via at least one linking group; and
at least one pendant fatty chain chosen from alkyl chains and alkenyl chains, wherein the at least one pendant fatty chain is bonded to the polymer skeleton via at least one linking group,
wherein the at least one first polymer and the at least one second polymer are each present in a sufficient amount to render the composition stable, and
wherein the at least one liquid fatty phase is structured by at least one of the at least one first polymer and the at least one second polymer.

4. The composition according to claim 1, wherein the at least one first polymer or at least one second polymer comprises at least one polyamide block or is a polyamide polymer.

5. The composition according to claim 1, wherein the at least one first polymer or at least one second polymer comprises at least one terminal fatty chain.

6. The composition according to claim 5, wherein the at least one terminal fatty chain is chosen from alkyl chains and alkenyl chains, each comprising at least four carbon atoms.

7. The composition according to claim 6, wherein the alkyl chains and the alkenyl chains each comprise from 12 to 68 carbon atoms.

8. The composition according to claim 1, wherein the at least one linking group of the at least one first polymer is an ester group present in a proportion ranging from 15% to 40% of the total number of all ester and heteroatom groups in the at least one first polymer.

9. The composition according to claim 1, wherein the at least one linking group of the at least one first polymer is an ester group present in a proportion ranging from 20% to 35% of the total number of all ester and heteroatom groups in the at least one first polymer.

10. The composition according to claim 1, wherein in the at least one first polymer, the percentage of the total number of fatty chains ranges from 40% to 98% relative to the total number of all repeating units and fatty chains in the at least one first polymer.

11. The composition according to claim 1, wherein in the at least one first polymer, the percentage of the total number of fatty chains ranges from 50% to 95% relative to the total number of all repeating units and fatty chains in the at least one first polymer.

12. The composition according to claim 1, wherein the at least one hydrocarbon-based repeating unit of the first polymer comprises from 2 to 80 carbon atoms.

13. The composition according to claim 1, wherein the at least one heteroatom of the at least one hydrocarbon-based repeating unit of the at least one first polymer is chosen from nitrogen, sulfur, and phosphorus.

14. The composition according to claim 13, wherein the at least one heteroatom is a nitrogen atom.

15. The composition according to claim 1, wherein the at least one heteroatom of the at least one first polymer, taken together with at least one oxygen atom, forms an amide group.

16. The composition according to claim 1, wherein the at least one first polymer is chosen from polyamide polymers of formula (III): 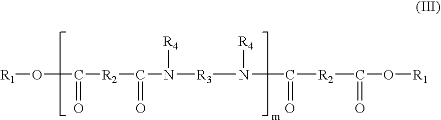
wherein:
m is an integer which represents the number of amide units such that the number of ester groups present in the at least one polyamide polymer ranges from 10% to 50% of the total number of all the ester groups and all the amide groups comprised in the at least one polyamide polymer;
R₁, which are identical or different, are each independently chosen from alkyl groups comprising at least 4 carbon atoms and alkenyl groups comprising at least 4 carbon atoms;
R₂, which are identical or different, are each independently chosen from C₄ to C₄₂ hydrocarbon-based groups, with the proviso that at least 50% of all R₂ groups are chosen from C₃₀ to C₄₂ hydrocarbon-based groups;
R₃, which may be identical or different, are each independently chosen from organic groups comprising at least two carbon atoms, in addition to hydrogen atoms, and optionally comprising at least one atom chosen from oxygen atoms and nitrogen atoms; and
R₄, which are identical or different, are each independently chosen from hydrogen atoms, C₁ to C₁₀ alkyl groups and a direct bond to at least one group chosen from R₃ and another R₄ such that when the at least one group is chosen from another R₄, the nitrogen atom to which both R₃ and R₄ are bonded forms part of a heterocyclic structure defined in part by R₄—N—R₃, with the proviso that at least 50% of all R₄ are chosen from hydrogen atoms.

17. The composition according to claim 16, wherein m is an integer ranging from 1 to 5.

18. The composition according to claim 16, wherein R., which are identical or different, are each chosen from C₁₆ to C₂₂ alkyl groups.

19. The composition according to claim 16, wherein R₂, which are identical or different, are each chosen from C₁₀ to C₄₂ hydrocarbon based groups, with the proviso that at least 50% of all R₂ are chosen from C₃₀ to C₄₂ hydrocarbon based groups.

20. The composition according to claim 16, wherein R₃, which are identical or different, are each chosen from C₂ to C₁₂ hydrocarbon-based groups.

21. The composition according to claim 16, wherein R₄, which are identical or different, are each chosen from hydrogen atoms.

22. The composition according to claim 1, wherein the at least one first polymer has a weight-average molecular mass ranging from 1000 to 30,000.

23. The composition according to claim 1, wherein the at least one first polymer has a softening point greater than 50° C. and less than 150° C.

24. The composition according to claim 1, wherein the at least one first polymer is present in the composition in an amount ranging from 0.5% to 80% by weight relative to the total weight of the composition.

25. The composition according to claim 1, wherein the at least one second polymer is a resin composition prepared by reacting components comprising dibasic acid, diamine, polyol and monoalcohol, wherein:
i) at least 50 equivalent percent of the dibasic acid comprises polymerized fatty acid;
ii) at least 50 equivalent percent of the diamine comprises ethylenediamine;
iii) 10 to 60 equivalent percent of the total of the hydroxyl and amine equivalents provided by diamine, polyol and monoalcohol are provided by monoalcohol; and
iv) no more than 50 equivalent percent of the total of the hydroxyl and amine equivalents provided by diamine, polyol and monoalcohol are provided by polyol.

26. The composition of claim 25, wherein polymerized fatty acid comprises at least 75 equivalent percent of the acid equivalents of the dibasic acid.

27. The composition of claim 25, wherein polymerized fatty acid comprises at least 90 equivalent percent of the acid equivalents of the dibasic acid.

28. The composition of claim 25, wherein ethylenediamine comprises at least 75 equivalent percent of the amine equivalents from diamine.

29. The composition of claim 25, wherein polymerized fatty acid comprises at least 75 equivalent percent of the acid equivalents of the dibasic acid, and ethylenediamine comprises at least 75 equivalent percent of the amine equivalents of diamine.

30. The composition of claim 25, wherein the monoalcohol reactant comprises an alcohol of the formula R₃—OH and R₃ is a hydrocarbon group.

31. The composition of claim 30, wherein R₃ is chosen from alkyl and aralkyl groups.

32. The composition of claim 25, wherein the monoalcohol is chosen from decanol, 1-dodecanol, tetradecanol, hexadecanol, octadecanol (stearyl alcohol), behenyl alcohol and linear wax alcohols comprising from 22 to 70 carbon atoms.

33. The composition of claim 25, wherein the polyol is of the formula R₄—(OH)ₙ wherein R₄ is an n-valent organic group.

34. The composition of claim 33, wherein R₄ is a C₂-C₂₀ organic group without hydroxyl substitution.

35. The composition of claim 33, wherein n is chosen from 2, 3, 4, 5 and 6.

36. The composition of claim 25, wherein the polyol is chosen from ethylene glycol, propylene glycol, butylene glycol, glycerol, trimethylolpropane, pentaerythritol, neopentyl glycol, tris(hydroxylmethyl)methanol, d i-pentaerythritol, and tri-pentaerthyritol.

37. The composition of claim 25, wherein the amine equivalents from diamine equal 0.3 to 0.75 of the total amine and hydroxyl equivalents provided by diamine, polyol and mono-alcohol.

38. The composition of claim 25, wherein the hydroxyl equivalents from polyol equal 0.05 to 0.45 of the total amine and hydroxyl equivalents provided by diamine, polyol and mono-alcohol.

39. The composition of claim 25, wherein the hydroxyl equivalents from mono-alcohol equal 0.20 to 0.45 of the total amine and hydroxyl equivalents provided by diamine, polyol and mono-alcohol.

40. The composition of claim 25, wherein the dibasic acid reactant comprises co-diacid chosen from 1,4-cyclohexane dicarboxylic acid, isophthalic acid, adipic acid, azeleic acid, sebacic acid, and dodecandioic acid.

41. The composition of claim 25, wherein the diamine reactant comprises co-diamine chosen from 1,6-hexanediamine, xylenediamine, 1,2-propanediamine, 2-methylpentamethylenediamine, and 1,12-dodecanediamine.

42. The composition according to claim 1, wherein the at least one second polymer is a structuring polymer for the liquid fatty phase.

43. The composition according to claim 1, wherein the polymer skeleton of the at least one second polymer is a polyamide skeleton.

44. The composition according to claim 1, wherein the at least one second polymer comprises at least one terminal fatty chain bonded to the polymer skeleton via at least one linking group chosen from single bonds and urea, urethane, thiourea, thiourethane, thioether, thioester, ether, amide, tertiary amide or secondary amide groups.

45. The composition according to claim 44, wherein the at least one second polymer comprises at least one terminal fatty chain bonded to the polymer skeleton via at least one ether group or polyether group.

46. The composition according to claim 44, wherein the at least one second polymer comprises at least one terminal fatty chain bonded to the polymer skeleton via at least one tertiary amide group.

47. The composition according to claim 44, wherein the second polymer is chosen from polyamide polymers of formula (II) 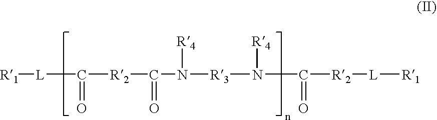
wherein:
n is an integer from 1 to 30,
R′₁, which are identical or different, are each independently a fatty chain chosen from alkyl groups comprising at least one carbon atom and alkenyl groups comprising at least two carbon atoms;
R′₂, which are identical or different, are each independently chosen from C₁ to C₅₂ hydrocarbon diradicals;
R′₃, which may be identical or different, are each independently chosen from organic groups comprising at least two carbon atoms, in addition to hydrogen atoms, and optionally comprising at least one atom chosen from oxygen atoms and nitrogen atoms;
R′₄, which are identical or different, are each independently chosen from hydrogen atoms, C₁ to C₁₀ alkyl groups and a direct bond to at least one group chosen from R′₃ and another R′₄, such that when the at least one group is chosen from another R′₄, the nitrogen atom to which both R′₃ and R′₄ are bonded forms part of a heterocyclic structure defined in part by R′₄—N—R′₃, with the proviso that at least 50% of all R′₄ are chosen from hydrogen atoms; and
L represents a linking group, which is substituted by at least one R′₁ group as defined above.

48. The composition according to claim 47, wherein the at least one second polymer is chosen from polyamide polymers of formula (II) wherein L is a group of formula:

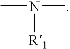

49. The composition according to claim 48, wherein the at least one second polymer is chosen from polyamide polymers of formula (IIa): 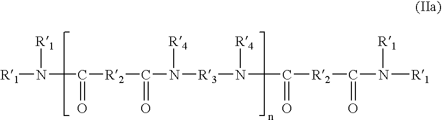
wherein:
n designates a number of repeating units such that terminal amide groups comprise from 10% to 50% of the total amide groups;
R′₁ at each occurrence is independently chosen from a C₁₋₂₂ hydrocarbon group;
R′₂ at each occurrence is independently chosen from a C₂₋₄₂ hydrocarbon group;
R′₃ at each occurrence is independently chosen from an organic group comprising at least two carbon atoms in addition to hydrogen atoms, and optionally comprising at least one atom chosen from oxygen and nitrogen atoms; and
R′₄ at each occurrence is independently chosen from hydrogen, C₁₋₁₀ alkyl and a direct bond to R′₃ or another R′₄ such that the N atom to which R′₃ and R′₄ are both bonded is part of a heterocyclic structure defined in part by R′₄—N—R′₃.

50. The composition of claim 49, wherein R′₁, at each occurrence, is independently chosen from a C₄-C₂₂ hydrocarbon group.

51. The composition of claim 49, wherein R′₂, at each occurrence, is independently chosen from a C₄-C₄₂ hydrocarbon group.

52. The composition of claim 49, wherein R′₃, at each occurrence, is independently chosen from a C₂-C₄₂ hydrocarbon group, where at least 50% of the R′₂ groups comprise from 30 to 42 carbon atoms.

53. The composition according to claim 47, wherein the at least one second polymer is chosen from polyamide polymers of formula (II), wherein L is a group of formula:
Z [symbol] R′₅—O [symbol] ₓ
wherein
R′₅ is chosen from C₂-C₆ hydrocarbon diradicals;
Z is chosen from O and NH; and
x is an integer ranging from 2 to 100.

54. The composition according to claim 53, wherein the at least one second polymer is chosen from polyamide polymers of formula (IIb): 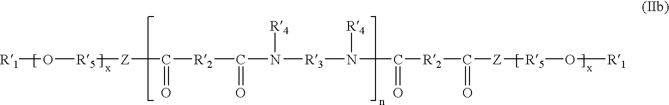
wherein
R′₁, which are identical or different, are each independently chosen from Cₗ-C₂₂ alkyl and C₁-C₂₂ alkylene radicals;
Z are chosen from O and NH;
x is an integer ranging from 2 to 100;
R′₂, which are identical or different, are each independently chosen from C₂ to C₅₂ hydrocarbon diradicals, wherein at least 50% of the R′₂ comprise at least 34 carbon atoms;
R′₃, which are identical or different, are each independently chosen from C₂-C₃₆ hydrocarbon diradicals and C₄-C₁₀₀ polyether diradicals;
R′₄, which are identical or different, are each independently chosen from hydrogen atoms, C₁ to C₁₀ alkyl groups and a direct bond to at least one group chosen from R′₃ and another R′₄ such that when at least one group is chosen from another R₄, the nitrogen atom to which both R′₃ and R′₄ are bonded forms part of a heterocyclic structure defined in part by R′₄—N—R′₃, with the proviso that at least 50% of all R′₄ are chosen from hydrogen atoms;
R′₅ are chosen from C₂-C₆ hydrocarbon diradicals; and
n is an integer ranging from 1 to 10.

55. The composition according to claim 54, wherein Z is NH.

56. The composition according to claim 54, wherein R′₅ is a C₂ hydrocarbon diradical.

57. The composition according to claim 54, wherein at least 80% of the R′₂ diradicals comprise at least 34 carbon atoms.

58. The composition according to claim 54, wherein the R′₃ group is a polyether.

59. The composition according to claim 1, wherein the at least one first polymer is present in the composition in an amount ranging from 0.5% to 80% by weight relative to the total weight of the composition.

60. The composition according to claim 59, wherein the at least one first polymer is present in the composition in an amount ranging from 2% to 60% by weight relative to the total weight of the composition.

61. The composition according to claim 60, wherein the at least one first polymer is present in the composition in an amount ranging from 5% to 40% by weight relative to the total weight of the composition.

62. The composition according to claim 61, wherein the at least one first polymer is present in the composition in an amount ranging from 5% to 25% by weight relative to the total weight of the composition.

63. The composition according to claim 62, wherein the at least one first polymer is present in the composition in an amount ranging from 5% to 15% by weight relative to the total weight of the composition.

64. The composition according to claim 1, wherein the at least one second polymer is present in the composition in an amount ranging from 0.5% to 80% by weight relative to the total weight of the composition.

65. The composition according to claim 64, wherein the at least one second first polymer is present in the composition in an amount ranging from 2% to 60% by weight relative to the total weight of the composition.

66. The composition according to claim 65, wherein the at least one second first polymer is present in the composition in an amount ranging from 5% to 40% by weight relative to the total weight of the composition.

67. The composition according to claim 66, wherein the at least one second first polymer is present in the composition in an amount ranging from 5% to 25% by weight relative to the total weight of the composition.

68. The composition according to claim 67, wherein the at least one second first polymer is present in the composition in an amount ranging from 5% to 15% by weight relative to the total weight of the composition.

69. A composition according to claim 1, wherein the ratio of the at least one first polymer to the at least one second polymer ranges from 1/10 to 10/1.

70. A composition according to claim 69, wherein the ratio of the at least one first polymer to the at least one second polymer ranges from 1/5 to 5/1.

71. A composition according to claim 70, wherein the ratio of the at least one first polymer tothe at least one second polymer ranges from 1/2 to 4/1.

72. A composition according to claim 71, wherein the ratio of the at least one first polymer to the at least one second polymer is 1/1.

73. A composition according to claim 70, wherein the ratio of the at least one first polymer to the at least one second polymer ranges from 4/1 to 5/1.

74. A composition according to claim 71, wherein the ratio of the at least one first polymer and the at least one second polymer is 3/1.

75. A composition according to claim 1, wherein the at least one first polymer has a softening point from 70° C. to 100° C.

76. A composition according to claim 1, wherein the at least one second polymer has a softening point from 80° C. to 110° C.

77. A composition according to claim 1, wherein the composition is free of wax.

78. The composition according claim 1, wherein the at least one liquid fatty phase of the composition comprises at least one oil chosen from at least one polar oil and at least one apolar oil, and wherein the at least one oil has an affinity for the at least one first polymer.

79. The composition according to claim 78, wherein the at least one polar oil is chosen from:
hydrocarbon-based plant oils with a high content of triglycerides comprising fatty acid esters of glycerol, wherein the fatty acids comprise chains comprise from 4 to 24 carbon atoms, said chains being optionally chosen from linear and branched, and saturated and unsaturated chains;
synthetic oils or esters of formula R₅COOR₆, wherein R₅ is chosen from linear and branched fatty acid residues comprising from 1 to 40 carbon atoms, and R₆ is chosen from alkyl groups comprising from 1 to 40 carbon atoms, with the proviso that R₅±R_{6≧10;}
synthetic ethers comprising from 10 to 40 carbon atoms;
C₈ to C₂₆ fatty alcohols; and

80. C₈ to C₂₆ fatty acids. The composition according to claim 78, wherein the at least one apolar oil is chosen from:
silicone oils chosen from volatile and non-volatile, linear and cyclic polydimethylsiloxanes that are liquid at room temperature;
polydimethylsiloxanes comprising alkyl or alkoxy groups, wherein each alkyl or alkoxy group is independently chosen from being pendant and being at the end of the silicone chain, and wherein the groups each comprise from 2 to 24 carbon atoms;
phenylsilicones; and
hydrocarbons chosen from linear and branched, volatile and non-volatile hydrocarbons of synthetic and mineral origin.

81. The composition according to claim 1, wherein the composition comprises at least one coloring agent chosen from pigments and dyes.

82. The composition according to claim 1, wherein the composition is in the form of a cosmetic composition.

83. The composition according to claim 82, wherein the composition is in the form of a treating shampoo product, a hair conditioning product, a sunscreen product, or a skin care formula.

84. The composition according to claim 82, wherein the composition is in the form of a colored make-up product for the skin, an eyeshadow, a concealer, an eyeliner, a make-up for the body, a nail varnish, a make-up for the lips, a make-up for eyelashes, and a make-up for the eyebrows.

85. The composition according to claim 84, wherein a make-up for the lips is chosen from lipgloss and lipstick.

86. The composition according to claim 1, wherein the composition is in a form chosen from an emulsion, an oil-in-water emulsion, a water-in-oil emulsion, an oil-in-water-in-oil emulsion, a water-in-oil-in-water emulsion, a solid gel, a supple gel, and an anhydrous composition.

87. A make-up composition comprising
i) at least one liquid fatty phase:
ii) at least one first polymer comprising
a) a polymer skeleton which comprises at least one hydrocarbon-based repeating unit comprising at least one heteroatom, and
b) at least one terminal fatty chain that is bonded to the polymer skeleton via at least one ester linking group; and
iii) at least one second polymer comprising
a) a polymer skeleton which comprises at least one hydrocarbon-based repeating unit comprising at least one heteroatom, and
b) at least one terminal fatty chain that is bonded to the polymer skeleton via at least one linking group different from an ester group.

88. The composition according to claim 87, wherein the composition is in the form of a lipstick.

89. A method for care or make up of a keratin material chosen from lips, skin, and keratinous fibers, comprising applying to the keratin material a cosmetic composition comprising
i) at least one liquid fatty phase,
ii) at least one first polymer comprising a polymer skeleton which comprises at least one hydrocarbon-based repeating unit comprising at least one heteroatom, and
iii) at least one second polymer, different from the first polymer, comprising
a) a polymer skeleton which comprises at least one hydrocarbon-based repeating unit comprising at least one heteroatom, and
b) at least one of:
at least one terminal fatty chain chosen from alkyl chains and alkenyl chains, wherein the at least one terminal fatty chain is bonded to the polymer skeleton via at least one linking group; and
at least one pendant fatty chain chosen from alkyl chains and alkenyl chains, wherein the at least one pendant fatty chain is bonded to the polymer skeleton via at least one linking group,
wherein the at least one first polymer and the at least one second polymer are each present in a sufficient amount to render the composition stable, and
wherein the at least one liquid fatty phase is structured by at least one of the at least one first polymer and the at least one second polymer.

90. A method for providing stability to a cosmetic composition comprising at least one liquid fatty phase, comprising including in the cosmetic composition:
ii) at least one first polymer comprising
a) a polymer skeleton which comprises at least one hydrocarbon-based repeating unit comprising at least one heteroatom, and
b) at least one of:
at least one terminal fatty chain chosen from alkyl chains and alkenyl chains, wherein the at least one terminal fatty chain is bonded to the polymer skeleton via at least one linking group; and
at least one pendant fatty chain chosen from alkyl chains and alkenyl chains, wherein the at least one pendant fatty chain is bonded to the polymer skeleton via at least one linking group, and
iii) at least one second polymer, different from the first polymer, comprising
a) a polymer skeleton which comprises at least one hydrocarbon-based repeating unit comprising at least one heteroatom, and
b) at least one of:
at least one terminal fatty chain chosen from alkyl chains and alkenyl chains, wherein the at least one terminal fatty chain is bonded to the polymer skeleton via at least one linking group; and
at least one pendant fatty chain chosen from alkyl chains and alkenyl chains, wherein the at least one pendant fatty chain is bonded to the polymer skeleton via at least one linking group.
